(19) United States
(12) Patent Application Publication
(10) Pub. No.: US 2018/0057873 A1
(43) Pub. Date: Mar. 1, 2018

(54) METHODS FOR PERFORMING SPATIAL PROFILING OF BIOLOGICAL MATERIALS

(71) Applicant: CENTRILLION TECHNOLOGY HOLDINGS CORPORATION, Grand Cayman (KY)

(72) Inventors: Wei ZHOU, Saratoga, CA (US); Janet WARRINGTON, Los Altos, CA (US)

(21) Appl. No.: 15/563,015

(22) PCT Filed: Apr. 18, 2016
(86) PCT No.: PCT/US16/28118

§ 371 Date: Sep. 29, 2017

Related U.S. Application Data
(60) Provisional application No. 62/149,385, filed on Apr. 17, 2015.
(60) Provisional application No. 62/148,747, filed on Apr. 17, 2015.
(60) Provisional application No. 62/148,758, filed on Apr. 17, 2015.

(51) Int. Cl. C12Q 1/68; C12N 15/10

(52) U.S. Cl. CPC C12Q 1/6874; C12N 15/1068; C12N 15/1065

(57) ABSTRACT

Provided herein are methods and compositions for profiling the spatial distribution of a wide variety of biological molecules in a sample. The methods and compositions are suited for spatial labeling and sequencing of biological molecules (e.g., nucleic acids, proteins) in a biological sample.

### CROSS-REFERENCE

This application claims priority to U.S. Provisional Application No. 62/148,747, filed on Apr. 17, 2015, U.S. Provisional Application No. 62/148,758, filed on Apr. 17, 2015, and U.S. Provisional Application No. 62/149,385, filed on Apr. 17, 2015; each of which is herein incorporated by reference in its entirety.

### BACKGROUND OF THE INVENTION

Determining the spatial distribution of biological molecules can be of great importance for life sciences research, molecular diagnostics and many other applications. In addition to understanding the gene expression profile of a particular cell or tissue, spatial information of biological molecules (e.g., nucleic acids, proteins) within the cell or tissue may also provide valuable information. For example, gene expression profiling of cancer cells can be important for monitoring cancer therapy.

### SUMMARY OF THE INVENTION

In one aspect, a method is provided comprising: a) contacting a biological sample comprising a plurality of biological molecules with a spatial barcode array, wherein the spatial barcode array comprises a plurality of oligonucleotides attached thereto, wherein each of the plurality of oligonucleotides comprises a barcode sequence that identifies a location of the plurality of oligonucleotides on the spatial barcode array; b) attaching the plurality of oligonucleotides to the plurality of biological molecules to generate a plurality of tagged biological molecules; c) sequencing at least a portion of the plurality of tagged biological molecules; and d) determining a location of the plurality of biological molecules within the biological sample based on the barcode sequence attached to the tagged biological molecules.

In some cases, the plurality of biological molecules are DNA. In some cases, the plurality of biological molecules are RNA. In some cases, the RNA is mRNA. In some cases, the method further comprises, prior to c) reverse transcribing the mRNA to cDNA. In some cases, the plurality of oligonucleotides comprise a polyT sequence. In some cases, the attaching comprises ligating the plurality of oligonucleotides to the plurality of biological molecules. In some cases, the attaching comprises annealing the plurality of oligonucleotides to the plurality of biological molecules. In some cases, the method further comprises, after the annealing, extending the plurality of oligonucleotides, using the plurality of biological molecules as a template, to generate a sequencing library. In some cases, the method further comprises, prior to the sequencing, amplifying the plurality of tagged biological molecules to generate an amplified sequencing library. In some cases, each of the plurality of oligonucleotides comprises one or more adaptor sequences. In some cases, each of the plurality of oligonucleotides comprises one or more primer sequences. In some cases, the barcode sequence identifies an x and y coordinate for the plurality of biological molecules within the biological sample. In some cases, the biological sample is a tissue section or a transfer of a tissue section. In some cases, the method further comprises performing a)-d) on a plurality of consecutive tissue sections to generate a three-dimensional profile of the biological molecules within the biological sample. In some cases, the barcode sequence further identifies a z coordinate for the plurality of biological molecules within the three-dimensional profile. In some cases, the tissue section is a biopsy sample. In some cases, the tissue section is a formalin-fixed paraffin-embedded (FFPE) tissue section. In some cases, the barcode sequence of each of the plurality of oligonucleotides is different. In some cases, the barcode sequence is indicative of the location of an oligonucleotide of the plurality of oligonucleotides on the spatial barcode array to within 2 μm. In some cases, the barcode sequence is indicative of the location of an oligonucleotide of the plurality of oligonucleotides on the spatial barcode array to within 1 μm. In some cases, the barcode sequence is indicative of the location of an oligonucleotide of the plurality of oligonucleotides on the spatial barcode array to within 0.5 μm. In some cases, the barcode sequence is indicative of the location of an oligonucleotide of the plurality of oligonucleotides on the spatial barcode array to within 0.2 μm. In some cases, the barcode sequence is indicative of the location of an oligonucleotide of the plurality of oligonucleotides on the spatial barcode array to within 0.1 μm. In some cases, the spatial barcode array comprises a solid support.

In another aspect, a method is provided comprising: a) contacting a biological sample comprising a plurality of biological molecules with a spatial barcode array, wherein the spatial barcode array comprises a plurality of oligonucleotides attached thereto, wherein each of the plurality of oligonucleotides comprises a barcode sequence that identifies a location of the plurality of oligonucleotides on the spatial barcode array; b) attaching the plurality of oligonucleotides to a signal sequence associated with each of the plurality of biological molecules to generate a plurality of tagged signal sequences; c) sequencing at least a portion of the plurality of tagged signal sequences; and d) determining a location of the plurality of biological molecules within the biological sample based on the barcode sequence attached to the plurality of tagged signal sequences. In some cases, the plurality of biological molecules are proteins. In some cases, the signal sequence is a tag oligonucleotide. In some cases, the signal sequence is conjugated to an affinity molecule. In some cases, the affinity molecule is an antibody, an aptamer, a peptide or a peptidomimetic. In some cases, the method further comprises, prior to b), contacting the biological sample with a plurality of affinity molecules, each of which are conjugated to a signal sequence, under conditions that permit binding of the plurality of affinity molecules to the plurality of biological molecules. In some cases, at least a portion of the signal sequence identifies the affinity molecule conjugated thereto. In some cases, each affinity molecule is conjugated to a different signal sequence. In some cases, the attaching comprises ligating the plurality of oligonucleotides to the signal sequence associated with each of the plurality of biological molecules. In some cases, the attaching comprises annealing the plurality of oligonucleotides to the plurality of signal sequences associated with each of the plurality of biological molecules. In some cases, the method further comprises, after the annealing, extending the plurality of oligonucleotides, using signal sequence associated with each of the plurality of biological molecules as a template to generate a sequencing library. In some cases, the method further comprises, prior to the sequencing, amplifying the plurality of tagged signal sequences to generate an amplified sequencing library. In some cases, each of the plurality of oligonucleotides comprises one or more adaptor sequences. In some cases, each of the plurality of oligonucleotides comprises one or more primer sequences. In some cases, the barcode sequence identifies an x and y coordinate for the plurality of biological molecules within the biological sample. In some cases, the biological sample is a tissue section or a transfer of a tissue section. In some cases, the method further comprises performing a)-d) on a plurality of consecutive tissue sections to generate a three-dimensional profile of the plurality of biological molecules within the biological sample. In some cases, the barcode sequence further identifies a z coordinate for the plurality of biological molecules within the three-dimensional profile. In some cases, the tissue section is a biopsy sample. In some cases, the tissue section is a formalin-fixed paraffin-embedded (FFPE) tissue section. In some cases, the barcode sequence of each of the plurality of oligonucleotides is different. In some cases, the barcode sequence is indicative of the location of an oligonucleotide of the plurality of oligonucleotides on the spatial barcode array to within 2 μm. In some cases, the barcode sequence is indicative of the location of an oligonucleotide of the plurality of oligonucleotides on the spatial barcode array to within 1 μm. In some cases, the barcode sequence is indicative of the location of an oligonucleotide of the plurality of oligonucleotides on the spatial barcode array to within 0.5 μm. In some cases, the barcode sequence is indicative of the location of an oligonucleotide of the plurality of oligonucleotides on the spatial barcode array to within 0.2 μm. In some cases, the barcode sequence is indicative of the location of an oligonucleotide of the plurality of oligonucleotides on the spatial barcode array to within 0.1 μm. In some cases, the spatial barcode array comprises a solid support.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a tissue section or a transfer of a tissue section placed on a spatially encoded oligonucleotide array as provided herein.

FIG. 2 illustrates a non-limiting example of a set up for performing a cross surface reaction as described herein.

FIG. 3 illustrates a non-limiting example of one aspect of the invention as described herein. FIG. 3A illustrates an example of attaching a spatial barcode oligonucleotide to an mRNA molecule as described herein. FIG. 3B illustrates an example of generating a sequencing library.

FIG. 4 illustrates a non-limiting example of one aspect of the invention as described herein. FIG. 4A illustrates an example of preparation of spatial barcode oligonucleotides suitable for performing the methods described herein. FIG. 4B illustrates one example of attaching a spatial barcode oligonucleotide to a nucleic acid molecule by ligation. FIG. 4C illustrates an example of attaching an adaptor to a spatially tagged nucleic acid molecule. FIG. 4D illustrates an example of generating a sequencing library.

FIG. 5 illustrates a non-limiting example of one aspect of the invention as described herein. FIG. 5A illustrates an example of attaching a spatial barcode oligonucleotide to an oligonucleotide-tagged antibody for spatially profiling biological molecules. FIG. 5B illustrates an example of generating a sequencing library.

### DETAILED DESCRIPTION OF THE INVENTION

In one aspect of the invention, methods are provided for profiling the spatial distribution of a wide variety of biological molecules. In some aspects, the methods involve the use of a spatial barcode array comprising a plurality of spatial barcodes. The spatial barcode array can be used to detect the molecular distribution of biological samples. The spatial barcodes may be oligonucleotides that include a sequence of nucleotides that can be ascertained to provide information as to the spatial location of the barcode on the array. In some cases, the spatial barcode array can be used to detect the distribution of biological molecules present within the biological sample. In some cases, the biological molecules are nucleic acid molecules such as DNA or RNA. In other cases, the biological molecules are proteins.

In one aspect, a method is provided comprising: a) contacting a biological sample comprising a plurality of biological molecules with a spatial barcode array, wherein the spatial barcode array comprises a plurality of oligonucleotides attached thereto, wherein each of the plurality of oligonucleotides comprises a barcode sequence that identifies a location of the plurality of oligonucleotides on the spatial barcode array; b) attaching the plurality of oligonucleotides to the plurality of biological molecules to generate a plurality of tagged biological molecules; c) sequencing at least a portion of the plurality of tagged biological molecules; and d) determining a location of the plurality of biological molecules within the biological sample based on the barcode sequence attached to the tagged biological molecules.

In another aspect, a method is provided comprising: a) contacting a biological sample comprising a plurality of biological molecules with a spatial barcode array, wherein the spatial barcode array comprises a plurality of oligonucleotides attached thereto, wherein each of the plurality of oligonucleotides comprises a barcode sequence that identifies a location of the plurality of oligonucleotides on the spatial barcode array; b) attaching the plurality of oligonucleotides to a signal sequence associated with each of the plurality of biological molecules to generate a plurality of tagged signal sequences; c) sequencing at least a portion of the plurality of tagged signal sequences; and d) determining a location of the plurality of biological molecules within the biological sample based on the barcode sequence attached to the plurality of tagged signal sequences.

In some cases, the biological sample can be a tissue sample. The tissue sample can be, for instance, a tissue section, such as a section of a cancer biopsy sample. Tissue sections can be obtained using, for example, microtomy or cryomicrotomy techniques. In other examples, the biological sample can be a monolayer of cells, such as one grown under tissue culture conditions. In some cases, the biological sample is a fixed sample. In some cases, the fixed sample is a formalin-fixed paraffin embedded (FFPE) tissue sample

The biological sample may be contacted with the spatial barcode array. For example, a section of a tissue sample can be laid down on the spatial barcode array such that the biological molecules within the tissue sample are in direct contact with the spatial barcode array. The biological molecules of the biological sample may then be reacted with the spatial barcodes directly or through one or more transfer or reaction steps that preserve the spatial location of the biological molecules to generate spatially tagged biological molecules.

In some aspects, the biological molecules are nucleic acid molecules, such as mRNA. In this example, the spatial barcodes may be attached to the nucleic acid molecules, for example, by ligation or by primer extension. In other aspects, the biological molecules are proteins. In this example, the spatial barcodes may be attached to an oligonucleotide tag associated with the proteins. In one particular example, an oligonucleotide tag can be conjugated to, for example, an affinity molecule (e.g., antibody) that binds to the proteins. The spatial barcodes may then be attached to the oligonucleotide tag when the oligonucleotide tag is in close proximity to the spatial barcodes (i.e., when the antibody binds to the protein).

The spatially tagged biological molecules may then be used as a template to prepare a sequencing library. The sequencing library may be analyzed to decode the original distribution of biological molecules in the biological sample. Such analysis may involve the identification and/or quantification of the biological molecules. For example, the location of the biological molecules within the biological sample may be determined based on the sequence of the spatial barcodes attached thereto. In addition, the identity of the biological molecule may be determined based on the sequence of at least a portion of the biological molecule or the signal sequence associated with the biological molecule.

In some examples, multiple sections of a biological tissue are assayed for the spatial detection of biological molecules. In some examples, the multiple sections are consecutive sections such that a three-dimensional distribution of the biological molecules can be determined.

Three Dimensional Gene Expression Profiling

In certain aspects, the methods described herein provide for the spatial detection of nucleic acid molecules within a biological sample (e.g., a tissue section). It is to be understood that essentially any naturally occurring nucleic acid molecule can be assayed using the methods provided herein. In some cases, the nucleic acids are DNA. In other cases, the nucleic acids are mRNA. In some cases, the mRNA is reverse transcribed to cDNA prior to sequencing. The biological sample may be treated, prior to practicing the methods provided herein, to preserve the spatial distribution of nucleic acid molecules. The distribution of the nucleic acid molecules can then be ascertained utilizing the methods provided herein.

In some cases, the biological sample is a tissue section. The tissue section can be obtained from a tissue sample, for example, a biopsy sample. In some cases, the tissue sample is fixed prior to sectioning. In other cases, the tissue sample is fixed after sectioning. Methods of fixing tissue samples are known to one skilled in the art, and essentially any fixation method may be used so long as the method preserves the spatial distribution of the nucleic acid molecules and is compatible with the methods provided herein. In some cases, the tissue sample is frozen prior to sectioning. The tissue sample may be sectioned by any method, for example, by microtomy or cryomicrotomy. In some cases, multiple, consecutive tissue sections can be obtained to produce a series of tissue sections that can be profiled to produce a three-dimensional spatial profile of the nucleic acid molecules.

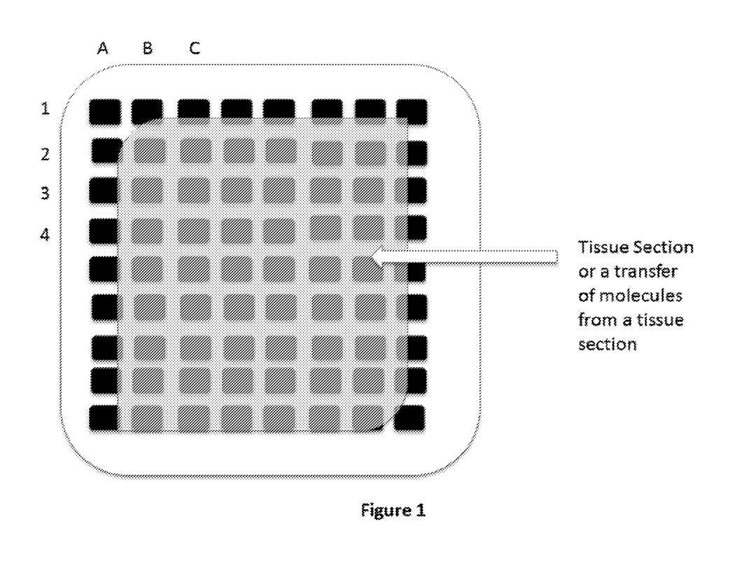

In some aspects, multiple tissue sections may be analyzed to generate three dimensional gene expression profiles. In some cases, the tissue sections are consecutive tissue sections. In some cases, the mRNA molecules can be located in a three dimensional space. The three dimensional profiling, or RNA-CT technology, may be useful for, example, analyzing cancer tissue gene expression. FIG. 1 is a schematic depicting a tissue section or a transfer of biological molecules placed on or contacted on top of a spatial DNA barcode array. Each feature of the array may contain an oligonucleotide barcode that can identify the location of the feature (e.g., x, y location). Multilayer x, y coordinates can be determined to provide a three-dimensional identification (e.g., x, y, and z location). In some cases, the barcode sequence can identify the z coordinate of the biological molecule within the three-dimensional profile.

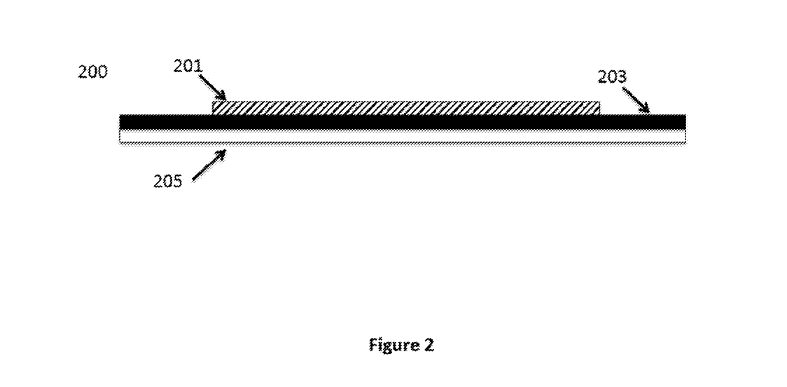

After obtaining the biological sample, it can be pressed or laid against or on top of a spatial barcode array. In some cases, the biological sample may be immobilized on a soft matrix (e.g., polyacrylamide layer) to facilitate close contact of the biological sample with the surface of the array. The close contact of the biological sample with the surface of the array may allow the spatial barcodes present on the surface of the array to contact the nucleic acid molecules. FIG. 2 depicts a non-limiting example of a set up for performing a cross surface reaction 200. In some cases, a biological sample (e.g., a tissue section or a transfer of the tissue section) 201 is placed on top of a spatial barcode array 205. In this example, the spatial barcode array may contain a soft matrix layer 203 (e.g., a polyacrylamide layer) that facilitates close contact of the biological sample 201 with the spatial barcode array 205.

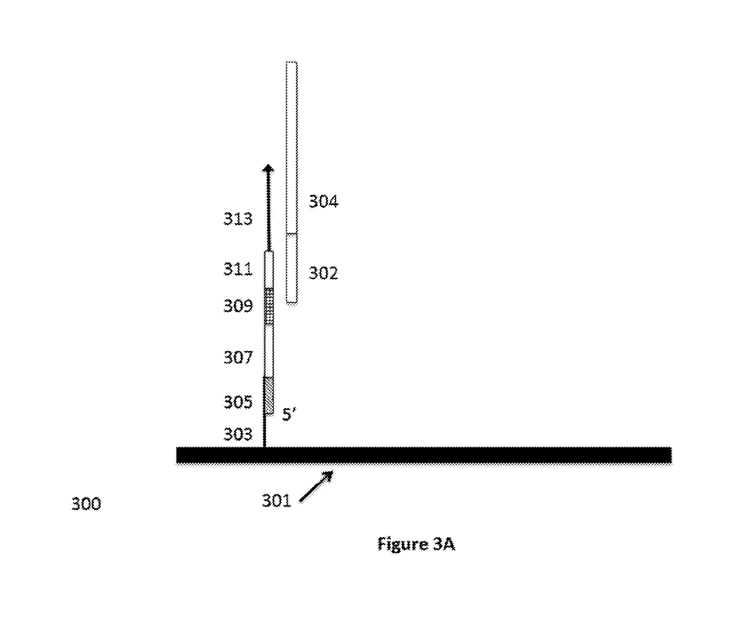

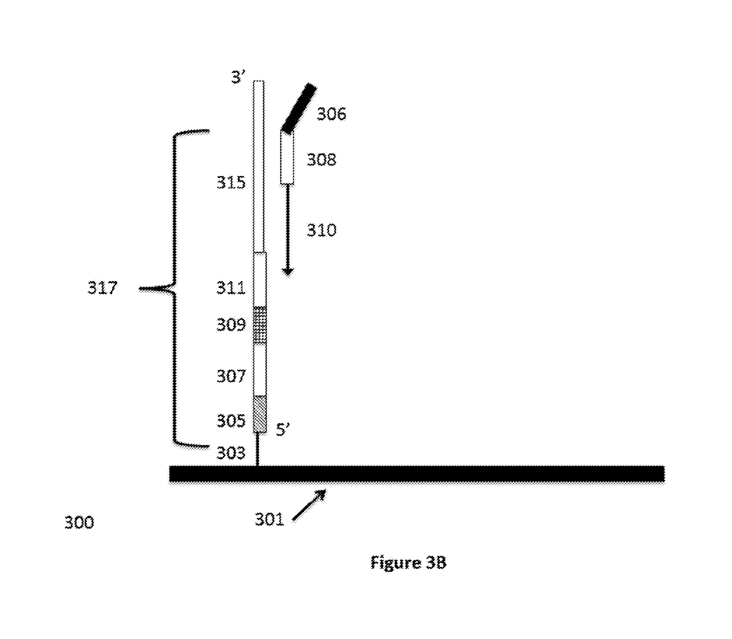

FIG. 3A depicts a non-limiting example of the structure of a spatial barcode oligonucleotide as described herein. The spatial barcode 307 may encode the x and y coordinates (and optionally, z) of the feature position on the spatial barcode array. In this example structure, appropriate sequence library adaptors including an amplification library and a sequencing primer binding site can be built in 305, 309. Specific adaptor sequences will be dependent upon the sequencing system, for example, the adaptor sequences on a sequencing flow cell. Any adaptor sequence may be built into the spatial barcode oligonucleotide.

In certain aspects, the spatial barcode oligonucleotides are attached to the nucleic acid molecules present in the biological sample. Attaching the spatial barcode oligonucleotides to the nucleic acid molecules may include ligation or incorporation by primer extension. In the example of primer extension, the spatial barcode oligonucleotides may include a primer sequence that is capable of hybridizing to a portion of the target nucleic acid molecules. The primer sequence may be specific for a target sequence or may be a random sequence. In cases where the nucleic acid molecules are mRNA molecules, the primer sequence may include a polyT sequence that can hybridize with the polyA tail of the mRNA molecules. The primer sequence may then act as a primer for an extension reaction. The spatial barcode oligonucleotide can then be extended using the nucleic acid molecules present in the biological sample as a template, and generating an extended spatial barcode oligonucleotide that includes the spatial barcode, the primer sequence and a sequence complementary to the nucleic acid molecules.

FIG. 3 depicts a non-limiting example of spatially barcoding mRNA molecules 304 within a biological sample. The spatial barcode oligonucleotides may be attached to the spatial barcode array 300 by a linker 303. The spatial barcode oligonucleotides may further include one or more adaptors 305, 309. The adaptors may include, for example, one or more sequencing adaptors, one or more primer sequences, one or more additional barcode sequences, and the like. Each spatial barcode oligonucleotide will include a unique spatial barcode sequence 307 that identifies the location of the spatial barcode oligonucleotide on the spatial barcode array. The spatial barcode oligonucleotide may include a polyT sequence 311 that can hybridize to the polyA tail 302 of the mRNA molecules 304. A reverse transcriptase and an appropriate buffer may be applied between the biological sample and the spatial barcode array. The structure may then be incubated at a temperature that allows a reverse transcription reaction 313 to take place. This example produces a library of spatially tagged cDNA molecules, each with a spatial barcode appended thereto. After the reverse transcription reaction, the tissue section may optionally be removed and the resulting spatially tagged cDNA molecules 317 may be amplified, as shown in FIG. 3B. The spatially tagged cDNA molecules 317 may be hybridized with a random hexamer or other suitable primer (e.g., a target-specific primer) 308 and amplified 310. The amplification step may involve dNTPs and a DNA polymerase. In some cases, the primers may include one or more sequencing adaptors or sample indexes 306.

The library of spatially tagged cDNA molecules can then be sequenced using any known sequencing method and the identification and spatial distribution of the original mRNA molecule can be interrogated utilizing at least a portion of the cDNA sequence and the spatial barcode attached thereto. In some cases, prior to sequencing, the sequencing library can be amplified using one or more primers and a DNA polymerase to generate an amplified library of spatially tagged oligonucleotides. The amplified library of spatially tagged oligonucleotides can then be sequenced as described above. The resulting sequences can be analyzed to generate a quantitative gene expression profile that includes spatial information of the original mRNA molecule in the biological sample.

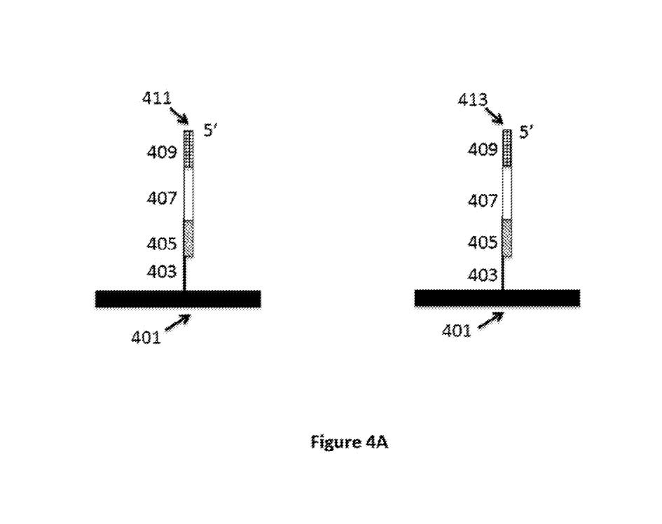

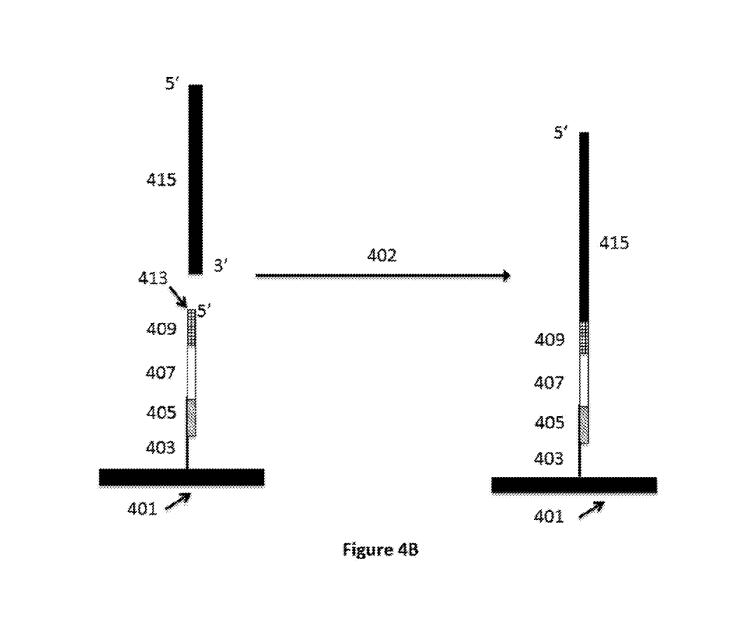

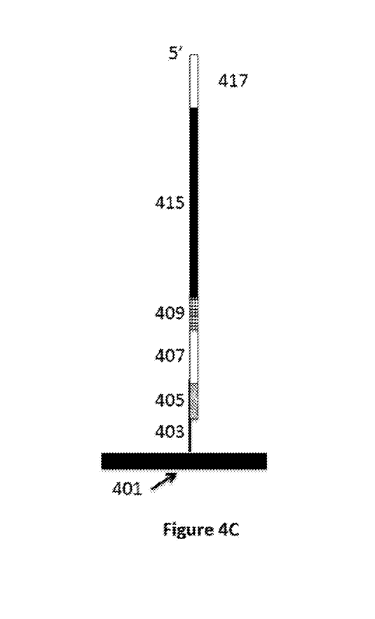

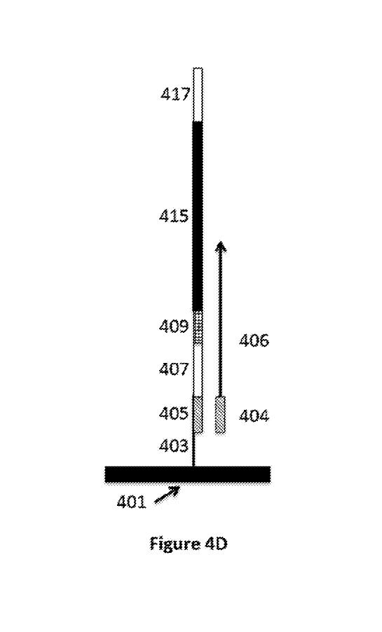

In other aspects, spatial barcodes can be ligated to the ends of the nucleic acid molecules. Any method of ligating the ends of nucleic acid molecules can be utilized. In some cases, the spatial barcode oligonucleotides are ligated to the ends of single-stranded RNA or DNA molecules. FIG. 4A depicts a non-limiting example of a spatial barcode oligonucleotide structure that may be utilized for spatially barcoding mRNA molecules. In this example, the spatial barcode oligonucleotides are synthesized on the array 401 by 3′ to 5′ synthesis. The spatial barcode oligonucleotides may be attached to the spatial barcode array by a linker 403. The spatial barcode oligonucleotides may include one or more adaptors 405, 409, for example, one or more sequencing adaptors, one or more primer sequences, one or more additional barcode sequences, and the like. Each of the spatial barcode oligonucleotides will include a spatial barcode sequence 407 that identifies the location of the spatial barcode oligonucleotide on the spatial barcode array. Each of the spatial barcode oligonucleotides are phosphorylated at the 5′ end of the molecules 411. In this example, the 5′end of the spatial barcodes can be ligated to the 3′ end of mRNA molecules using, for example, T4 RNA ligase. In cases where the ligase requires a pre-adenylated 5′ end (e.g., T4 RNA ligase), the 5′ end of the spatial barcode oligonucleotides can be enzymatically adenylated prior to ligation 413. FIG. 4B depicts a non-limiting example of ligating a spatial barcode oligonucleotide to an RNA molecule. In this example, the pre-adenylated 5′ end of the spatial barcode oligonucleotide 413 is ligated using T4 RNA ligase 402 to the 3′ end of an RNA molecule 415 present in the biological sample, thereby generating a spatially tagged RNA molecule. The spatially tagged RNA molecule can be further appended with one or more RNA adaptors as depicted in FIG. 4C. The one or more RNA adaptors 417 can be ligated to the 5′ end of the spatially tagged RNA molecule using, for example, T4 RNA ligase. The spatially tagged RNA molecules can then be used as a template for the construction of a sequencing library. As depicted in FIG. 4D, a primer can be hybridized to the spatially tagged RNA molecule 404 and the primer can be extended 406 using the spatially tagged RNA molecule as a template. The resulting cDNA molecules can be further amplified to generate the spatially tagged sequencing library. In some cases, a reverse transcriptase is utilized to reverse transcribe the RNA molecule into cDNA, followed by an optional amplification step with a DNA polymerase. In other cases, a polymerase that has both reverse transcriptase and DNA polymerase activities may be utilized (e.g., PyroPhage® DNA Polymerase). The reaction conditions may be similar to those found in, e.g., Chen et al. Plant Methods 2012, 8:41, incorporated herein by reference.

In some aspects, ribosomal RNA can be removed or reduced by a variety of methods known in the art before the library construction. Additionally or alternatively, library sequences derived from ribosomal RNA can be reduced by hybridizing ribosomal sequence specific probes with the library. Such probes can be labeled with biotin or other affinity groups and the hybridized sequences can be removed by binding with streptavidin-coated beads or surfaces.

The spatial barcode array may contain spatial barcodes wherein each feature or location on the array includes one distinct barcode sequence. In some cases, each location of the spatial barcode array is about 1 mm², about 2 mm², about 3 mm², about 4 mm², about 5 mm², about 6 mm², about 7 mm, about 8 mm², about 9 mm², about 10 mm², about 11 mm², about 12 mm², about 13 mm², about 14 mm², about 15 mm², about 16 mm², about 17 mm², about 18 mm², about 19 mm², about 20 mm² or greater than about 20 mm². The barcode sequence may be different in a certain edit distance, such as an edit distance of 4 to allow for error correction.

Profiling of Other Biological Molecules

In certain aspects, the distribution of other biological molecules including protein molecules can be analyzed utilizing the methods provided herein. These methods generally will involve the use of an affinity molecule that has binding capability to the biological molecule. For example, the affinity molecule can be an antibody or an antibody fragment. In other examples, the affinity molecule can be an aptamer. In other examples, the affinity molecule can be a peptide or a peptidomimetic. In yet other examples, the affinity molecule can be a ligand. Essentially any molecule can be used as the affinity molecule as long as the molecule has affinity for the biological molecules of interest in the biological sample. The affinity molecule can be specific, for example, an antibody that binds to a specific epitope on a protein molecule. In other examples, the affinity molecule may target a lipid or structural component of a cell (e.g., cytoskeleton).

The affinity molecule may be conjugated to a signal sequence which identifies the specific affinity molecule. In some cases, the signal sequence is an oligonucleotide tag. Any known chemistry may be utilized to conjugate an oligonucleotide to an affinity molecule. Each species of affinity molecule will have a unique signal sequence (e.g., oligonucleotide tag) such that the signal sequence can be sequenced to determine the identity of the affinity molecule to which it is conjugated to, and subsequently, can identify its target biological molecule. The signal sequence may be connected with adaptors that facilitate the subsequent sequencing library construction and connection with the spatial barcode oligonucleotides. The biological sample (e.g., tissue section) can be contacted with the signal sequence-conjugated affinity molecule under conditions in which the signal sequence-conjugated affinity molecule can bind to a target biological molecule in the biological sample. Once the signal sequence-conjugated affinity molecules are bound to their target biological molecules, the tissue section can be washed to remove any unbound affinity molecules and then the tissue section can be contacted with the spatial barcode array. In one example, the spatial barcode array may include a plurality of spatial barcode oligonucleotides, as described herein, that includes a sequence that can hybridize to the signal sequence (e.g., oligonucleotide tag) of the affinity molecule. The hybridized sequence of the spatial barcode oligonucleotide may then act as a primer for a subsequent primer extension reaction using the signal sequence as a template. In other cases, the signal sequence may include a primer sequence that can hybridize to the spatial barcode oligonucleotide and prime an extension reaction using the spatial barcode oligonucleotide as a template. In other cases, the spatial barcode array may include a plurality of spatial barcode oligonucleotides that can be ligated to the signal sequence of the affinity molecule.

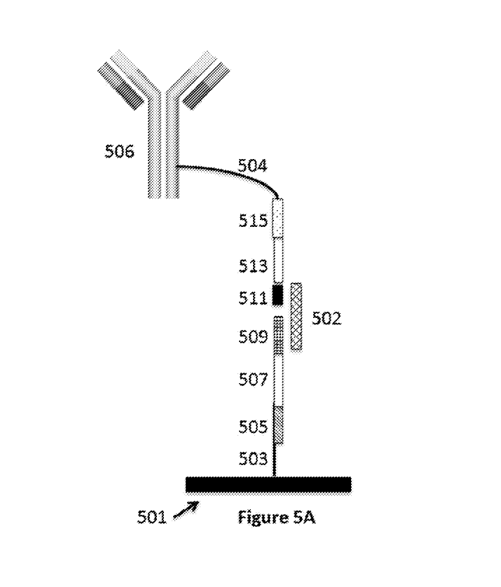

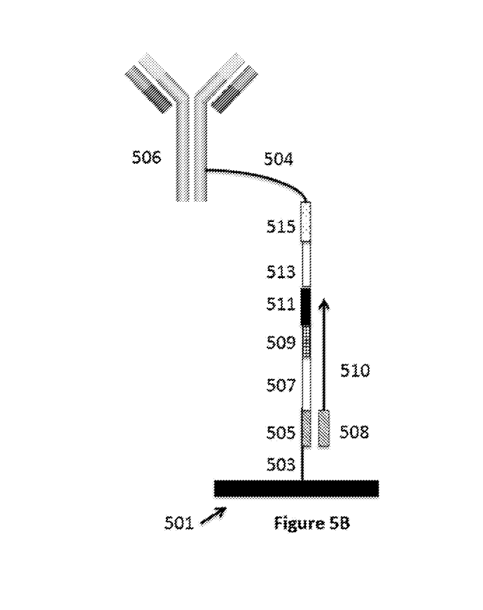

FIGS. 5A and 5B demonstrate an example of protein profiling of a biological sample utilizing the methods provided herein. As shown in FIG. 5A, a spatial barcode oligonucleotide arranged on a spatial barcode array is provided. The spatial barcode oligonucleotide may be attached to the spatial barcode array 501 by a linker 503. The spatial barcode oligonucleotide may further include one or more adaptors 505, 509. The adaptors may include, for example, one or more sequencing adaptors, one or more primer sequences, one or more additional barcode sequences, and the like. Each spatial barcode oligonucleotide will include a unique spatial barcode sequence 507 that identifies the location of the spatial barcode oligonucleotide on the spatial barcode array. A tissue section can be contacted with the spatial barcode array. The tissue section has been previously contacted with an antibody 506 that includes a signal sequence, in this example, an oligonucleotide tag 504, such that the antibody binds to a target protein molecule present within the biological sample. The oligonucleotide tag 504, as demonstrated in this example, may include one or more adaptor sequences 515, and a barcode sequence that includes information regarding the identity of the antibody bound thereto 513. The oligonucleotide tag can be attached to the spatial barcode oligonucleotide with the use of a helper oligonucleotide 502. The helper oligonucleotide may include a sequence complementary to a sequence on the spatial barcode oligonucleotide as well as a sequence complementary to a sequence on the oligonucleotide tag such that the helper oligonucleotide creates a bridge between the two molecules. The spatial barcode oligonucleotide and the oligonucleotide tag can be attached by ligating the ends together or by a gap filling reaction. A sequencing library can be generated by primer annealing and extension as depicted in FIG. 5B. The sequencing library can be optionally amplified by any known method.

Preservation of Spatial Relationaships

The molecular profiling methods of the invention may be performed on a tissue section or a sample derived from a tissue section where the spatial relationship of molecular distribution is reasonably preserved. For example, the molecules in a tissue section may be transferred from the tissue section to a surface and the surface can then be used for spatial profiling. For example, a tissue transfer may be a sample that preserves the positions of biological molecules of a tissue sample. The molecules in the transfer can be from the tissue section directly or by derivation such as template-directed DNA or RNA synthesis. In some cases, the target molecules do not need to be detected directly. For example, mRNA molecules may be hybridized with specific probes with identifying sequence. After washing away unbound probes, the identifying sequences or their derivatives may then be linked to spatial barcodes for profiling. cDNA molecules, for example, can be synthesized using a polyT sequence linked to barcodes. Alternatively, cDNA molecules may be synthesized first and then linked to spatial barcodes through cross surface reaction.

Biological Samples

A “nucleic acid molecule” or “nucleic acid” as referred to herein can be deoxyribonucleic acid (DNA) or ribonucleic acid (RNA) including known analogs or a combination thereof unless otherwise indicated. Nucleic acid molecules to be profiled herein can be obtained from any source of nucleic acid. The nucleic acid molecule can be single-stranded or double-stranded. In some cases, the nucleic acid molecule is DNA. The DNA can be mitochondrial DNA, cell-free DNA, complementary DNA (cDNA), or genomic DNA. In some cases, the nucleic acid molecule is genomic DNA (gDNA). The DNA can be plasmid DNA, cosmid DNA, bacterial artificial chromosome (BAC), or yeast artificial chromosome (YAC). The DNA can be derived from one or more chromosomes. For example, if the DNA is from a human, the DNA can derived from one or more of chromosome 1, 2, 3, 4, 5, 6, 7, 8, 9, 10, 11, 12, 13, 14, 15, 16, 17, 18, 19, 20, 21, 22, X, or Y. The RNA can include, but is not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long nc RNAs. In some aspects, the nucleic acid molecules are not purified prior to performing the methods provided herein. In some cases, the nucleic acid molecules are spatially distributed within a cell or tissue sample. In some cases, the nucleic acid molecules are profiled within the cell or tissue sample. The source of nucleic acid for use in the methods and compositions described herein can be a sample comprising the nucleic acid.

The terms “peptide” and “protein” may be used interchangeably herein to refer to polymers of amino acids of any length. A polypeptide can be any protein, peptide, protein fragment or component thereof. A polypeptide can be a protein naturally occurring in nature or a protein that is ordinarily not found in nature. A polypeptide can consist largely of the standard twenty protein-building amino acids or it can be modified to incorporate non-standard amino acids. A polypeptide can be modified, typically by the host cell, by e.g., adding any number of biochemical functional groups, including phosphorylation, acetylation, acylation, formylation, alkylation, methylation, lipid addition (e.g. palmitoylation, myristoylation, prenylation, etc) and carbohydrate addition (e.g. N-linked and O-linked glycosylation, etc). Polypeptides can undergo structural changes in the host cell such as the formation of disulfide bridges or proteolytic cleavage.

The biological sample can be derived from a non-cellular entity comprising polynucleotides (e.g., a virus) or from a cell-based organism (e.g., member of archaea, bacteria, or eukarya domains). In some cases, the sample is obtained from a swab of a surface, such as a door or bench top. In some cases, the sample is a tissue sample. The tissue sample may be a section of a tissue sample. In some cases, the tissue sample is obtained from a biopsy. The tissue sample can be frozen prior to profiling. In some cases, the tissue sample can be fixed, for example, with formalin or formaldehyde prior to performing the methods provided herein. In some cases, the tissue is embedded in an embedding medium suitable for performing any known tissue sectioning technique. In some cases, the tissue is embedded in paraffin wax. In one example, the tissue section is obtained from a formalin-fixed paraffin embedded (FFPE) tissue sample. In some cases, the FFPE tissue sample is deparaffinized prior to performing the methods described herein. In some cases, the structure and/or organization of the tissue or cell sample is maintained during the sample processing steps. In some cases, the tissue sample is a blood sample. In some cases, the sample is a cell sample such as a cell culture sample. In some cases, the sample includes cells in suspension. In this example, the suspended cells may be spun onto a slide or directly onto the spatial barcode array (e.g., using a cytospin). In some cases, the sample is a transfer of a tissue. For example, a tissue transfer may be a sample that preserves the positions of biological molecules of a tissue sample. The molecules in the transfer can be from the tissue section directly or by derivation such as template-directed DNA or RNA synthesis.

The biological sample can be from a subject, e.g., a plant, fungi, eubacteria, archeabacteria, protist, or animal. The subject can be an organism, either a single-celled or multi-cellular organism. The subject can be cultured cells, which can be primary cells or cells from an established cell line, among others. The sample can be isolated initially from a multi-cellular organism in any suitable form. The animal can be a fish, e.g., a zebrafish. The animal can be a mammal. The mammal can be, e.g., a dog, cat, horse, cow, mouse, rat, or pig. The mammal can be a primate, e.g., a human, chimpanzee, orangutan, or gorilla. The human can be a male or female. The sample can be from a human embryo or human fetus. The human can be an infant, child, teenager, adult, or elderly person. The female can be pregnant, suspected of being pregnant, or planning to become pregnant. In some cases, the sample is a single or individual cell from a subject and the biological molecules are derived from the single or individual cell. In some cases, the sample is an individual micro-organism, or a population of micro-organisms, or a mixture of micro-organisms and host cellular or cell free nucleic acids.

The biological sample can be from a subject (e.g., human subject) who is healthy. In some cases, the biological sample is taken from a subject (e.g., an expectant mother) at least 4, 5, 6, 7, 8, 9, 10, 11, 12, 13, 14, 15, 16, 17, 18, 19, 20, 21, 22, 23, 24, 25, or 26 weeks of gestation. In some cases, the subject is affected by a genetic disease, a carrier for a genetic disease or at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs).

The biological sample can be from a subject who has a specific disease, disorder, or condition, or is suspected of having (or at risk of having) a specific disease, disorder or condition. For example, the biological sample can be from a cancer patient, a patient suspected of having cancer, or a patient at risk of having cancer. The cancer can be, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. The sample can be from the cancer and/or normal tissue from the cancer patient. In some cases, the sample is a biopsy of a tumor.

The biological sample can be aqueous humour, vitreous humour, bile, whole blood, blood serum, blood plasma, breast milk, cerebrospinal fluid, cerumen, enolymph, perilymph, gastric juice, mucus, peritoneal fluid, saliva, sebum, semen, sweat, tears, vaginal secretion, vomit, feces, or urine. The biological sample can be obtained from a hospital, laboratory, clinical or medical laboratory. The sample can be taken from a subject.

The biological sample can be an environmental sample comprising medium such as water, soil, air, and the like. The biological sample can be a forensic sample (e.g., hair, blood, semen, saliva, etc.). The biological sample can comprise an agent used in a bioterrorist attack (e.g., influenza, anthrax, smallpox).

The biological sample can comprise nucleic acid. The biological sample can comprise protein. The biological sample can be a cell line, genomic DNA, cell-free plasma, formalin fixed paraffin embedded (FFPE) sample, or flash frozen sample. A formalin fixed paraffin embedded sample can be deparaffinized before performing the methods provided herein. The biological sample can be from an organ, e.g., heart, skin, liver, lung, breast, stomach, pancreas, bladder, colon, gall bladder, brain, etc.

The biological sample can be processed to render it competent for performing any of the methods provided herein. Examples of sample processing can include, without limitation, fixing the cells or tissue, embedding the cells or tissue in an embedding medium, sectioning the cells or tissue, and/or combining the sample with reagents for further nucleic acid processing. In some examples, the sample can be combined with a restriction enzyme, reverse transcriptase, or any other enzyme of nucleic acid processing.

Spatial Oligonucleotide Barcode Arrays

Techniques for preparing surfaces including oligonucleotide arrays with positional barcodes, preparing sequencing libraries, and other useful techniques are described in PCT Pub. No. WO/2015/085274, PCT Pub. No. WO/2015/085275, and PCT Pub. No. WO/2015/085268, each of which is incorporated by reference herein in its entirety.

To resolve the location of the biological molecules within the biological sample, a set of barcodes which uniquely define the position of the biological molecule on the chip can be provided. The barcodes can be accurately sequenced (e.g., GC content between 40% and 60%, no homopolymer runs longer than two, no self-complimentary stretches longer than 3, not present in human genome reference). Most importantly, to error-proof spatial addressability, each barcode may have an edit distance of four; that is, each barcode is at least four deletions, insertions, or substitutions away from any other barcode in the array. For example, a set of about 1.5 million 18-base barcodes can be employed. In some cases, the barcodes in the array have an edit distance of at least 1, 2, 3, 4, 5, 6, 7, 8, 9, 10 or greater than 10.

The spatial barcode array may comprise a plurality of oligonucleotides. In some cases, the oligonucleotides on the spatial barcode array may include one or more barcodes. In some cases, the one or more barcodes comprises a spatial barcode. The term “spatial barcode oligonucleotide” may refer to an oligonucleotide that includes a spatial barcode and any number of additional nucleic acid features (e.g., adaptors, primers, and the like). The term “oligonucleotide” can refer to a nucleotide chain, typically less than 200 residues long, e.g., between 15 and 100 nucleotides long. The oligonucleotide can comprise at least or about 1, 2, 3, 4, 5, 6, 7, 8, 9, 10, 15, 20, 25, 30, 35, 40, 45, or 50 bases. The oligonucleotides can be from about 3 to about 5 bases, from about 1 to about 50 bases, from about 8 to about 12 bases, from about 15 to about 25 bases, from about 25 to about 35 bases, from about 35 to about 45 bases, or from about 45 to about 55 bases. The oligonucleotide (also referred to as “oligo”) can be any type of oligo (e.g., primer). In some cases, the oligos are 5′-acrydite-modified oligos. The oligos can be coupled to the polymer coatings as provided herein on surfaces as provided herein. The oligonucleotides can comprise cleavable linkages. Cleavable linkages can be enzymatically cleavable. Oligonucleotides can be single- or double-stranded. The terms “primer” and “oligonucleotide primer” can refer to an oligonucleotide capable of hybridizing to a complementary nucleotide sequence. The term “oligonucleotide” can be used interchangeably with the terms “primer,” “adapter,” and “probe.” The term “polynucleotide” can refer to a nucleotide chain typically greater than 200 residues long. Polynucleotides can be single- or double-stranded. The term “hybridization”/“hybridizing” and “annealing” can be used interchangeably and can refer to the pairing of complementary nucleic acids.

The term “barcode” can refer to a known nucleic acid sequence that allows some feature of a nucleic acid (e.g., oligo) with which the barcode is associated to be identified. In some cases, the feature of the oligonucleotide to be identified is the spatial position of each oligonucleotide on an array or chip. The term “spatial barcode” may refer to a known nucleic acid sequence that allows the location of a biological molecule with which the barcode is associated to be resolved. A barcode can be a spatial barcode. The barcode or spatial barcode may be associated with an oligonucleotide as described herein (e.g., a spatial barcode oligonucleotide). The barcodes can be designed for precision sequence performance, e.g., GC content between 40% and 60%, no homo-polymer runs longer than two, no self-complementary stretches longer than 3, and be comprised of sequences not present in a human genome reference. A barcode sequence can be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence can be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence can be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. An oligonucleotide (e.g., primer or adapter) can comprise about, more than, less than, or at least 1, 2, 3, 4, 5, 6, 7, 8, 9, or 10 different barcodes. Barcodes can be of sufficient length and comprise sequences that can be sufficiently different to allow the identification of the spatial position of each biological molecule based on barcode(s) with which each biological molecule is associated. In some cases, each barcode is, for example, four deletions or insertions or substitutions away from any other barcode in an array. The oligonucleotides in each array spot on the barcoded oligonucleotide array can comprise the same barcode sequence and oligonucleotides in different array spots can comprise different barcode sequences. The barcode sequence used in one array spot can be different from the barcode sequence in any other array spot. Alternatively, the barcode sequence used in one array spot can be the same as the barcode sequence used in another array spot, as long as the two array spots are not adjacent. Barcode sequences corresponding to particular array spots can be known from the controlled synthesis of the array. Alternatively, barcode sequences corresponding to particular array spots can be known by retrieving and sequencing material from particular array spots.

Array Surface Preparation

The methods and compositions provided in this disclosure can comprise preparing a surface for generating an array. In some cases, the array is an array of oligonucleotides (oligonucleotide array or oligo array). The preparation of the surface can comprise creating a polymer coating on the surface. The surface can comprise glass, silica, titanium oxide, aluminum oxide, indium tin oxide (ITO), silicon, polydimethylsiloxane (PDMS), polystyrene, polycyclic olefins, polymethylmethacrylate (PMMA), cyclic olefin copolymer (COC), other plastics, titanium, gold, other metals, or other suitable materials. The surface can be flat or round, continuous or non-continuous, smooth or rough. Examples of surfaces include flow cells, sequencing flow cells, flow channels, microfluidic channels, capillary tubes, piezoelectric surfaces, wells, microwells, microwell arrays, microarrays, chips, wafers, non-magnetic beads, magnetic beads, ferromagnetic beads, paramagnetic beads, superparamagnetic beads, and polymer gels.

In some cases, preparation of surfaces as described herein for the generation of oligonucleotide arrays as provided herein comprises bonding initiator species to the surface. In some cases, the initiator species comprises at least one organosilane. In some cases, the initiator species comprises one or more surface bonding groups. In some cases, the initiator species comprises at least one organosilane and the at least one organosilane comprises one or more surface bonding groups. The organosilane can comprise one surface-bonding group, resulting in a mono-pedal structure. The organosilane can comprise two surface-bonding groups, resulting in a bi-pedal structure. The organosilane can comprise three surface-bonding groups, resulting in a tri-pedal structure. The surface bonding group can comprise MeO₃Si, (MeO)₃Si, (EtO)₃Si, (AcO)₃Si, (Me₂N)₃Si, and/or (HO)₃Si. In some cases, the surface bonding group comprises MeO₃Si. In some cases, the surface bonding group comprises (MeO)₃Si. In some cases, the surface bonding group comprises (EtO)₃Si. In some cases, the surface bonding group comprises (AcO)₃Si. In some cases, the surface bonding group comprises (Me₂N)₃Si. In some cases, the surface bonding group comprises (HO)₃Si. In some cases, the organosilane comprises multiple surface bonding groups. The multiple surface bonding groups can be the same or can be different. In some cases, the initiator species comprises at least one organophosphonic acid, wherein the surface bonding group comprises (HO)₂P(═O). The organophosphonic acid can comprise one surface-bonding group, resulting in a mono-pedal structure. The organophosphonic acid can comprise two surface-bonding groups, resulting in a bi-pedal structure. The organophosphonic acid can comprise three surface-bonding groups, resulting in a tri-pedal structure.

In some cases, a surface as provided herein comprises a surface-bound initiator species as provided herein for the generation of oligo arrays comprises a surface coating or functionalization. The surface coating or functionalization can be hydrophobic or hydrophilic. The surface coating can comprise a polymer coating or polymer brush, such as polyacrylamide or modified polyacrylamide. The surface coating can comprise a gel, such as a polyacrylamide gel or modified polyacrylamide gel. The surface coating can comprise metal, such as patterned electrodes or circuitry. The surface coating or functionalization can comprise a binding agent, such as streptavidin, avidin, antibodies, antibody fragments, or aptamers. The surface coating or functionalization can comprise multiple elements, for example a polymer or gel coating and a binding agent. In some cases, preparation of surfaces as described herein for the generation of oligonucleotide arrays as provided herein comprises forming a polymer coating on the surface-bound initiator species. The surface bound initiator species can be any surface bound initiator species known in the art. In some cases, the surface bound initiator species comprises an organosilane as provided herein. The organosilane can comprise one or more surface bonding groups as described herein. In some cases, the organosilane comprises at least two surface bonding groups. The presence of two or more surface bonding groups can serve to increase the stability of an initiator species-polymer coating complex. The one or more surface bonding groups can be any surface bonding group as provided herein. The resulting polymer coatings can comprise linear chains. The resulting polymer coatings can comprise chains that are branched. The branched chains can be lightly branched. A lightly branched chain can comprise less than or about 1, 2, 3, 4, 5, 6, 7, 8, 9, or 10 branches. The polymer coatings can form polymer brush thin-films. The polymer coatings can include some cross-linking. The polymer coatings can form a graft structure. The polymer coatings can form a network structure. The polymer coatings can form a branched structure. The polymers can comprise homogenous polymers. The polymers can comprise block copolymers. The polymers can comprise gradient copolymers. The polymers can comprise periodic copolymers. The polymers can comprise statistical copolymers.

In some cases, the polymer coating formed on the surface bound initiator species comprises polyacrylamide (PA). The polymer can comprise polymethylmethacrylate (PMMA). The polymer can comprise polystyrene (PS). The polymer can comprise polyethylene glycol (PEG). The polymer can comprise polyacrylonitrile (PAN). The polymer can comprise poly(styrene-r-acrylonitrile) (PSAN). The polymer can comprise a single type of polymer. The polymer can comprise multiple types of polymer. The polymer can comprise polymers as described in Ayres, N. (2010). Polymer brushes: Applications in biomaterials and nanotechnology Polymer Chemistry, 1(6), 769-777, or polymers as described in Barbey, R., Lavanant, L., Paripovic, D., Schüwer, N., Sugnaux, C., Tugulu, S., & Klok, H. A. (2009) Polymer brushes via surface-initiated controlled radical polymerization: synthesis, characterization, properties, and applications. Chemical reviews, 109(11), 5437-5527, the disclosure of each of which is herein incorporated by reference in its entirety.

Polymerization of the polymer coating on the surface bound initiator species can comprise methods to control polymer chain length, coating uniformity, or other properties. The polymerization can comprise controlled radical polymerization (CRP), atom-transfer radical polymerization (ATRP), or reversible addition fragmentation chain-transfer (RAFT). The polymerization can comprise living polymerization processes as described in Ayres, N. (2010). Polymer brushes: Applications in biomaterials and nanotechnology Polymer Chemistry, 1(6), 769-777, or as described in Barbey, R., Lavanant, L., Paripovic, D., Schüwer, N., Sugnaux, C., Tugulu, S., & Klok, H. A. (2009) Polymer brushes via surface-initiated controlled radical polymerization: synthesis, characterization, properties, and applications. Chemical reviews, 109(11), 5437-5527, the disclosure of each of which is herein incorporated by reference in its entirety.

The polymer coating formed on a surface bound initiator species as provided herein can be of uniform thickness over the entire area of the polymer coating. The polymer coating formed on a surface bound initiator species as provided herein can be of varying thickness across the area of the polymer coating. The polymer coating can be at least 1 μm, 2 μm, 3 μm, 4 μm, 5 μm, 7 μm, 8 μm, 9 μm, 10 μm, 15 μm, 20 μm, 25 μm, 30 μm, 40 μm thick. The polymer coating may be at least 50 μm thick. The polymer coating may be at least 75 μm thick. The polymer coating may be at least 100 μm thick. The polymer coating may be at least 150 μm thick. The polymer coating may be at least 200 μm thick. The polymer coating may be at least 300 μm thick. The polymer coating may be at least 400 μm thick. The polymer coating may be at least 500 μm thick. The polymer coating may be between about 1 μm and about 10 μm thick. The polymer coating may be between about 5 μm and about 15 μm thick. The polymer coating may be between about 10 μm and about 20 μm thick. The polymer coating may be between about 30 μm and about 50 μm thick. The polymer coating may be between about 10 μm and about 50 μm thick. The polymer coating may be between about 10 μm and about 100 μm thick. The polymer coating may be between about 50 μm and about 100 μm thick. The polymer coating may be between about 50 μm and about 200 μm thick. The polymer coating may be between about 100 μm and about 30 μm thick. The polymer coating may be between about 100 μm and about 500 μm thick.

In some cases, physiochemical properties of the polymer coatings herein are modified. The modification can be achieved by incorporating modified acrylamide monomers during the polymerization process. In some cases, ethoxylated acrylamide monomers are incorporated during the polymerization process. The ethoxylated acrylamide monomers can comprise monomers of the form CH₂═CH—CO—NH(—CH₂—CH2-O—)ₙH. The ethoxylated acrylamide monomers can comprise hydroxyethyl acrylamide monomers. The ethoxylated acrylamide monomers can comprise ethylene glycol acrylamide monomers. The ethoxylated acrylamide monomers can comprise hydroxyethylmethacrylate (HEMA). The incorporation of ethoxylated acrylamide monomers can result in a more hydrophobic polyacrylamide surface coating. In some cases, phosphorylcholine acrylamide monomers are incorporated during the polymerization process. In some cases, betaine acrylamide monomers are incorporated during the polymerization process.

The surfaces used for the transfer methods as provided herein (e.g., template surface and/or the recipient surface) can comprise a range of possible materials. In some cases, the surface comprises a polymer gel on a substrate, such as a polyacrylamide gel or a PDMS gel. In some cases, the surface comprises a gel without a substrate support. In some cases, the surface comprises a thin coating on a substrate, such as sub-200 nm coatings of polymer. In some cases, the surface comprises an uncoated substrate, such as glass or silicon.

The coatings and/or gels can have a range of thicknesses or widths. The gel or coating can have a thickness or width of about 0.0001, 0.00025, 0.0005, 0.001, 0.005, 0.01, 0.025, 0.05, 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, or 200 mm. The gel or coating can have a thickness or width of less than 0.0001, 0.00025, 0.0005, 0.001, 0.005, 0.01, 0.025, 0.05, 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, or 200 mm. The gel or coating can have a thickness or width of more than 0.0001, 0.00025, 0.0005, 0.001, 0.005, 0.01, 0.025, 0.05, 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, or 200 mm. The gel or coating can have a thickness or width of at least 0.0001, 0.00025, 0.0005, 0.001, 0.005, 0.01, 0.025, 0.05, 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, or 200 mm. The gel or coating can have a thickness or width of at most 0.0001, 0.00025, 0.0005, 0.001, 0.005, 0.01, 0.025, 0.05, 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, or 200 mm. The gel or coating can have a thickness or width of between 0.0001 and 200 mm, between 0.01 and 20 mm, between 0.1 and 2 mm, or between 1 and 10 mm. The gel or coating can have a thickness or width of from about 0.0001 to about 200 mm, about 0.01 to about 20 mm, about 0.1 to about 2 mm, or about 1 to about 10 mm. In some cases, the gel or coatings comprises a width or thickness of about 10 microns.

Gels and coatings can additionally comprise components to modify their physicochemical properties, for example, hydrophobicity. For example, a polyacrylamide gel or coating can comprise modified acrylamide monomers in its polymer structure such as ethoxylated acrylamide monomers, phosphorylcholine acrylamide monomers, and/or betaine acrylamide monomers.

Gels and coatings can additionally comprise markers or reactive sites to allow incorporation of markers. Markers can comprise oligonucleotides. For example, 5′-acrydite-modified oligonucleotides can be added during the polymerization process of a polyacrylamide gel or coating. Reactive sites for incorporation of markers can comprise bromoacetyl sites, azides, sites compatible with azide-alkyne Huisgen cycloaddition, or other reactive sites. Markers can be incorporated into the polymer coatings in a controlled manner, with particular markers located at particular regions of the polymer coatings. Markers can be incorporated into the polymer coatings at random, whereby particular markers can be randomly distributed throughout the polymer coatings.

In some cases, a surface with a gel coating can be prepared as follows: glass slides are cleaned (e.g., with NanoStrip solution), rinsed (e.g. with DI water), and dried (e.g. with N₂); the glass slide surface is functionalized with acrylamide monomers; a silanation solution is prepared (e.g., 5% by volume (3-acrylamidopropyl)trimethoxysilane in ethanol and water); the glass slide is submerged in the silanation solution (e.g. for 5 hours at room temperature), rinsed (e.g., with DI water), and dried (e.g. with N₂); a 12% acrylamide gel mix is prepared (e.g., 5 mL H₂O, 1 mg gelatin, 600 mg acrylamide, 32 mg bis-acrylamide); a 6% acrylamide gel mix is prepared (e.g., 50 μL 12% acrylamide gel mix, 45 μL DI water, 5 μL 5′-acrydite modified oligonucleotide primers (1 mM, vortexed to mix); 6% acrylamide gel mix is activated (e.g., 1.3 μL of 5% ammonium persulfate and 1.3 μL of 5% TEMED are each added per 100 μL of gel mix and vortexed); gel mix is applied to a surface (e.g. silanized functionalized glass slide surface), evenly spread (e.g. by pressing with a cover slip or by spin coating), and allowed to polymerize (e.g., 20 minutes at room temperature).

Photo-Directed Synthesis of the DNA Barcode Array

High-density oligonucleotide arrays of probe lengths up to 60 bp are commercially available, such as from Affymetrix, NimbleGen, and Agilent. With conventional contact lithography, stepwise misalignment can limit the achievable minimum feature size to about 1 to 2 μm, as demonstrated by the 20-mer oligo array synthesized using photolytic protecting group chemistry. Reduction of the feature size below 1 μm can be achieved through the combined use of projection lithography and contrast enhancing photoacid generating polymer films. Established steppers (e.g., ASML PAS5500) routinely print 5× reduced patterns in the sub-micron range with ±0.060 μm placement accuracy. In addition, the fully synthesized sequence can be ˜60 bases (˜20 base barcode, flanked by two ˜20 base universal adaptors). The top adaptor can eventually prime the immobilized DNA as discussed herein, while the bottom adaptor can serve as the first adaptor for NGS library preparation.

The feature size of arrays synthesized by techniques disclosed herein can be less than about 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, or 0.1 μm. The feature size of arrays synthesized by techniques disclosed herein can enable the identification of target nucleic acid positioning (e.g., the positioning of mutations, epigenetic modifications, or other features of a nucleic acid) to within about 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, or 0.1 μm.

Reversing the Oligo Orientation Via Gel Transfer

The standard phosphoramidite oligosynthesis using 5′ DMT protecting groups can result in oligos with the 3′ end attached to the surface. To serve as primers for polymerase extension on combed DNA, the oligo orientation may be reversed in some cases. A transfer method to copy the DNA array onto a second surface via face-to-face polymerase extension reaction is provided. A second surface with uniform coverage of immobilized primers complimentary to the bottom adaptor can be pressed into contact with the DNA array. The array sandwich can then be heated (e.g., to 55° C.), at which point polymerase (e.g., Bst polymerase in Thermopol PCR buffer) present at the interface can extend the primers hybridized to the bottom adaptor of the array creating a dsDNA molecular bridge between the surfaces. Upon physical separation of the arrays, the second surface can contain the complementary ssDNA barcode array with 5′ end attached to the surface and 3′ end available for polymerase extension. Since both the uniformly dispersed primer and the barcode oligos are tethered to their respective surfaces, the relative geographical locations of the transferred features will be maintained (in mirror image). To achieve intimate contact between the arrays, and thus uniform transfer over the full chip area, materials including PDMS and polyacrylamide have been evaluated.

The methods herein can also be used to generate oligo arrays with a desired orientation. In some cases, the methods for generating oligo arrays as provided herein on surfaces prepared for generating oligo arrays as provided herein are used to generate oligo arrays that are used as templates (i.e., template arrays) for the generation of one or more oligo arrays comprising oligos coupled thereto that are complementary to oligos on the template array. The oligo arrays comprising oligos coupled thereto that are complementary to a template array can be referred to as a recipient array (or alternatively, transfer array). The transfer or recipient oligo arrays can comprise oligos with a desired orientation. The transfer or recipient arrays can be generated from the template array using an array transfer process. In some cases, template oligo arrays with a desired feature (“spot”) density (e.g., feature or spot size of about 1 μm) are subjected to an array transfer process as provided herein in order to generate transfer or recipient oligo arrays with a desired orientation. The desired orientation can be a transfer or recipient oligo array that comprises oligos with the 5′ end of each oligo of the array attached to the array substrate. A template oligo array for generating the transfer or recipient oligo array with oligos in a desired orientation (i.e., 5′ end of each oligo of the array attached to the array substrate) can have the 3′ end of each oligo of the template array attached to the substrate. The array transfer process can be a face-to-face transfer process. In some cases, the face-to-face transfer process occurs by enzymatic transfer or enzymatic transfer by synthesis (ETS). In some cases, the face-to-face transfer process occurs by a non-enzymatic transfer process. The non-enzymatic transfer process can be oligonucleotide immobilization transfer (OIT).

The face-to-face gel transfer process (e.g., ETS or OIT) can significantly reduce the unit cost of fabrication while simultaneously flipping the oligo orientation (5′ immobilized) which can have assay advantages such as allowing for the enzymatic extension of the 3′ ends of the array bound oligos. Moreover, ETS or OIT can result in the transfer of a greater number or higher percentage of oligos of a desired or defined length (i.e., full-length oligo) from the template array to the recipient array. Subsequent amplification (e.g., amplification feature regeneration or AFR as provided herein) of the transferred full length product oligos on the recipient oligo arrays can allow the recipient oligo arrays to contain oligos comprising greater than 50 nucleotide bases without suffering from low yield or partial length products.

In some cases, a template and/or recipient array comprises polymers. The polymers can be aptamers or oligos. In some cases, a template or recipient array comprises oligos. A template or recipient array can have coupled to it at least 10, 20, 50, 100, 200, 500, 1,000, 2,000, 5,000, 10,000, 20,000, 50,000 or 100,000, 200,000, 500,000, 1,000,000, 2,000,000, 5,000,000, 10,000,000, 20,000,000, 100,000,000, 200,000,000, 500,000,000, or 1 billion template polymers (e.g., oligos). A template array can have template polymers arranged on it at a density of at least 10, 20, 50, 100, 200, 500, 1,000, 2,000, 5,000, 10,000, 20,000, 50,000 or 100,000 polymers (e.g., oligos) per square millimeter. The polymers (e.g., oligos) on a template or recipient array can be organized into spots, regions, or pixels. Polymers (e.g., oligos) in each spot or region can be identical to each other or related to each other (e.g., all or substantially all include a consensus or common sequence). Polymers (e.g., oligos) in each spot or region can be greater than 55, 60, 65, 70, 75, 80, 85, 90, 95, 99, or 99.9% identical to each other. The template or recipient array can comprise at least 1, 2, 3, 4, 5, 6, 7, 8, 9, 10, 100, 1000, 10,000, 100,000, 1,000,000, or 10,000,000 spots or regions. Each spot or region can have a size of at most about 1 cm, 1 mm, 500 μm, 200 μm, 100 μm, 10 μm, 9 μm, 8 μm, 7 μm, 6 μm, 5 μm, 4 μm, 3 μm, 2 μm, 1 μm, 800 nm, 500 nm, 300 nm, 100 nm, 50 nm, or 10 nm.

A recipient or transfer array generated as provided herein can comprise oligos that are fully complementary, fully identical, partially complementary, or partially identical in their sequence and/or number to oligos on the template array from which the recipient array was transferred. Partially complementary can refer to recipient arrays that have at least 40%, 45%, 50%, 55%, 60%, 65%, 70%, 75%, 80%, 85%, 90%, 95%, 96%, 97%, 98%, 99%, or 99.9% sequence complementarity. Partially identical can refer to recipient arrays that have at least 40%, 45%, 50%, 55%, 60%, 65%, 70%, 75%, 80%, 85%, 90%, 95%, 96%, 97%, 98%, 99%, or 99.9% sequence identity. A recipient array can have the same number of oligonucleotides as a template array and/or at least 40%, 45%, 50%, 55%, 60%, 65%, 70%, 75%, 80%, 85%, 90%, 95%, 96%, 97%, 98%, 99%, or 99.9% of the number of oligos as the template array from which the recipient array was transferred.

Array fabrication methods as provided herein can result in arrays having polymers (e.g. oligos) of the designed, desired, or intended length, which can be called full-length products. For example, a fabrication method intended to generate oligos with 10 bases can generate full-length oligos with 10 bases coupled to an array. Array fabrication processes can result in polymers (e.g. oligos) of less than the designed, desired, or intended length, which can be called partial-length products. The presence of partial-length oligos can be within a given feature (spot) or between features (spots). For example, a fabrication method intended to generate oligos with 10 bases can generate partial-length oligos with only 8 bases coupled to an array. That is, a synthesized oligo array can comprise many nucleic acids which are homologous or nearly homologous along their length, but which may vary in length from each other. Of these homologous or nearly homologous nucleic acids, those with the longest length can be considered full-length products. Nucleic acids with length shorter than the longest length can be considered partial-length products. Array fabrication methods provided herein can result in some full-length products (e.g., oligos) and some partial-length products (e.g., oligos) coupled to an array in a given feature (spot). Partial-length products coupled to a particular array or within a given feature can vary in length. Complementary nucleic acids generated from full-length products can also be considered full-length products. Complementary nucleic acids generated from partial-length products can also be considered partial-length products.

A transfer method as provided herein (e.g., ETS or OIT) can be used to increase or enrich the amount or percentage of full-length products (e.g., oligo) coupled to a recipient array surface. Array transfer (e.g., ETS or OIT) can result in a transfer or recipient array comprising at least, at most, more than, less than, or about 30%, 40%, 50%, 55%, 60%, 65%, 70%, 75%, 80%, 85%, 90%, 95%, 96%, 97%, 98%, 99%, or 99.9% transferred oligonucleotides that are 100% of the length of the respective oligonucleotide on a template array used to generate the transfer or recipient array. A transferred oligonucleotide that is 100% of the length (i.e., the same or identical length) of a template oligonucleotide can be referred to as full-length product (e.g., full-length product oligo). A template array fabricated by methods known in that art (e.g. spotting or in situ synthesis) can comprise about 20% oligonucleotides that are a desired length (i.e., full-length oligonucleotides) and about 80% oligonucleotides that are not a desired length (i.e., partial-length oligonucleotides). Transfer of the array generated by methods known in the art comprising about 20% full-length oligonucleotides and about 80% partial-length oligonucleotides using array transfer methods as provided herein can result in the generation of transfer or recipient arrays comprising at most about 20% full-length product oligos. In some cases, an array fabricated according to the methods herein has a greater percentage of oligonucleotides of a desired length (i.e., full length oligos) such that transfer of an array fabricated according to the methods herein using array transfer methods provided herein results in the generation of transfer or recipient arrays with a higher percentage of full-length product oligos as compared to fabrication and transfer methods known in the art.

In some cases, a transfer method provided herein (e.g., ETS or OIT) comprises generation of nucleic acid (e.g., oligo) sequences complementary to the template sequences. The transfer can occur by enzymatic replication (e.g., ETS) or by non-enzymatic physical transfer (e.g., OIT) of array components between array surfaces. The array surfaces can be any array surface as provided herein. The substrate of the template array and of the recipient array can be the same or can be different. The transfer can comprise fabrication of complementary sequences which are already attached to a recipient array; for example, primers bound to a recipient array, and are complementary to adaptors on the template array, can be extended using the template array sequences as templates to thereby generate a full length or partial length recipient array. Transfer can comprise fabrication of complementary sequences from a template array followed by attachment of the complementary sequences to a recipient array.

A transfer method as provided herein (e.g., ETS or OIT) can generate a recipient array such that the orientation of a template nucleic acid (e.g., oligo) relative to its coupled recipient array surface is preserved (e.g., the 3′ end of the template nucleic acid (e.g., oligo) is bound to the template array and the 3′ end of the transferred nucleic acid (e.g., oligo) complement is bound to the recipient array). Transfer can reverse the orientation of a nucleic acid relative to its coupled array surface (e.g., the 3′ end of the template nucleic acid is bound to the template array and the 5′ end of the transferred nucleic acid complement is bound to the recipient array).

Array transfer (e.g., ETS or OIT) can be performed multiple times. Array transfer (e.g., ETS or OIT) can be performed multiple times using the same template array. A template array of template polymers bound to a template substrate can be used for the production of at least 1, 2, 3, 4, 5, 6, 7, 8, 9, 10, 20, 30, 40, 50, 60, 70, 80, 90, 100, 500, 1,000, 5,000, 10,000, 50,000, or 100,000 recipient arrays. Array transfer can be performed multiple times in a series of transfers, using the transfer array from one array transfer as the template array for a subsequent transfer. For example, a first transfer can be performed from a template array with oligonucleotides bound to the array at their 3′ ends to a first transfer array with complementary oligonucleotides bound to the array at their 5′ ends, and a second transfer can be performed from the first transfer array (now serving as a template array) to a second transfer array with a higher percentage of full-length products and sequences matching the original template array than in recipient arrays generated using transfer techniques commonly used in the art while preserving the 5′-surface bound orientation. In some cases, the full-length product oligos on a recipient array generated using the array transfer methods provided herein (e.g., ETS or OIT) are further enriched through amplification of the full-length product oligos on the recipient array. Amplification can be conducted using the methods provided herein. The array transfer method can be a face-to-face enzymatic transfer method (e.g., ETS) or non-enzymatic (e.g., OIT) as provided herein.

In some cases, array transfer by ETS or OIT can be aided by the use of adaptor sequences on the template polymers (e.g., oligos). Polymers (e.g., oligos) can comprise a desired final sequence with the addition of one or more adaptor sequences. For example, a template oligonucleotide can comprise, in order, a 3′ end with a first adaptor sequence, a 5′ end with a second adaptor sequence, and a desired final sequence in the middle. The first and second adaptor sequences can be the same or can be different. In some cases, oligonucleotides in the same array spot comprise identical first and second adaptor sequences and final sequences, and oligonucleotides in different array spots comprise identical first and second adaptor sequences but different final sequences. Primers on a transfer/recipient array can be complementary to adaptor sequences, allowing hybridization between the primers and the template polymers (e.g., oligos). Such hybridization can aid in the transfer from one array to another.

Some or all adaptor sequences can be removed from transfer/recipient array polymers (e.g. transferred oligonucleotides) after transfer, for example by enzymatic cleavage, digestion, or restriction. Some or all adaptor sequences can be removed from transfer/recipient array polymers (e.g. transferred oligonucleotides) after transfer, for example by enzymatic cleavage, digestion, or restriction. For example, oligonucleotide array components can have adaptors removed via probe end clipping (PEC) by double-strand DNAse. Oligonucleotides complementary to the adaptor sequence can be added and hybridized to the array components. DNAse specific to double-stranded DNA can then be used to digest the oligonucleotides (see FIG. 10). Alternatively, one or more cleavable base, such as a dU, can be incorporated into the primer of the strand to be removed. The primer can then be nicked at the position next to the 3′-most base of the probe, and the nick site can be cut by an appropriate enzyme, such as Mung bean S1 or P1 nuclease. Many restriction enzymes and their associated restriction sites can also be used, including but not limited to EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, SacI, SalI, ScaI, SpeI, SphI, StuI, and XbaI. In some cases, the transfer process described above is repeated from the second surface (recipient surface) to a new, third surface containing primers (e.g., oligo) complementary to the top adaptor. Because only the full length oligos can have a complete top adaptor, only these can be copied onto the third array surface (i.e., new or third recipient or transfer array). The process can purify or enrich the full length oligos from the partial products, thus creating a high feature density, high quality full length oligo array. Purification or enrichment can mean the generation of a recipient array such that said recipient array has a greater percentage or number of oligos of a desired length (i.e. full-length) than the array used as a template for the generation of said recipient array. The full-length oligos can be oligos that contain all the desired features (e.g., adaptor(s), barcode(s), target nucleic acid or complement thereof, and/or universal sequence(s), etc.).

In some cases, array transfer can be aided by the flexibility or deformability of the array (e.g., template array) or of a surface coating on the array (e.g., template array). For example, an array (e.g., template array) comprising a polyacrylamide gel coating with coupled oligonucleotides can be used in array transfer (e.g., ETS, OIT). The deformability of the gel coating can allow for array components (oligos, reagents (e.g., enzymes)) to contact each other despite surface roughness. Surface roughness can be variability in the topography of the surface.

Array components can be amplified or regenerated by enzymatic reactions termed as amplification feature regeneration (AFR). AFR can be performed on template arrays and/or recipient arrays. AFR can be used to regenerate full-length oligos on an array (e.g., template and/or recipient) in order to ensure that each oligo in a feature (spot) on an array (e.g., template and/or recipient array) comprises desired components (e.g., adaptor(s), barcode(s), target nucleic acid or complement thereof, and/or universal sequence(s), etc.). AFR can be conducted on oligos comprising adaptor and/or primer binding sites (PBS) such that the oligos each comprise a first adaptor (or first PBS), probe sequence, and second adaptor (or second PBS). Preferably, the oligos in each feature on an array (e.g., template and/or recipient array) comprise two or more primer binding sites (or adaptor sequence). AFR can be performed used nucleic amplification techniques known in the art. The amplification techniques can include, but are not limited to, isothermal bridge amplification or PCR. For example, bridge amplification can be conducted on array (e.g., template and/or recipient array) component oligonucleotides via hybridization between adaptor sequences on the array (e.g., template and/or recipient array) components and surface-bound oligonucleotide primers, followed by enzymatic extension or amplification. Amplification can be used to recover lost array (e.g., template and/or recipient array) component density or to increase density of array (e.g., template and/or recipient array) components beyond their original density.

Immobilized oligos, nucleotides, or primers on an array as provided herein (e.g., template and/or recipient array) can be equal in length to each other or can have varying lengths. Immobilized oligos, nucleotides, or primers can comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, or 200 bases. In some cases, immobilized oligos, nucleotides, or primers are 71 bases long (71-mer).

The recipient surface of the transfer array can be brought into close proximity or contact with the template surface of the template array. In some cases, contact between the template array and the transfer array can be aided by the presence of a deformable coating, such as a polymer gel (e.g., polyacrylamide). The deformability of the coating can allow coupled polymers (e.g. oligonucleotides or primers) to come into close enough contact for hybridization to occur. The deformability of the coating can help overcome gaps due to surface roughness (e.g., surface topography variability) or other features that would otherwise prevent close enough contact for hybridization. An additional benefit of the deformable coating is that it can be pre-loaded with enzymatic reaction reagents, and thus serve as a reservoir for the interfacial reaction of enzymatic transfer by synthesis (ETS). One or both of the arrays can comprise a substrate with a gel coating with polymer molecules coupled to it. For example, the transfer array can comprise a substrate coupled to a polyacrylamide gel with oligonucleotide primers coupled to the gel. Surfaces and coatings are further discussed elsewhere in this disclosure.

### Enzymatic Transfer by Synthesis (ETS)

ETS can comprise a face-to-face polymerase extension reaction to copy one or more template oligos (e.g., DNA oligo) from a template oligo array onto a second surface (e.g., recipient array). A second surface (e.g., recipient array) with uniform coverage of immobilized primers complimentary to sequence on an oligo in the template oligo array (e.g., the bottom adaptor sequence in oligo arrays comprising adaptor sequence) can be pressed into contact with the template oligo (e.g., DNA oligo) array. A recipient array surface can comprise surface immobilized oligomers (oligos), nucleotides, or primers that are complementary, at least in part, to template nucleic acids or oligos on the template oligo array. In some cases, a transfer or recipient array comprises oligos that selectively hybridize or bind to aptamers on a template array. Immobilized oligos, nucleotides, or primers on a transfer or recipient array can be complementary to adaptor regions on template polymers (e.g. oligos).

The template nucleic acids (oligos) can hybridize with the immobilized primers or probes on the recipient surface, also called recipient primers or probes or transfer primers or probes. The hybridized complex (e.g., duplex) can be extended enzymatically such as, e.g., by DNA polymerase including but not limited to PolI, PolII, PolIII, Klenow, T4 DNA Pol, modified T7 DNA Pol, mutated modified T7 DNA Pol, TdT, Bst, Taq, Tth, Pfu, Pow, Vent, Pab, and pyrophage.

The transfer process can preserve the orientation of the oligonucleotides, i.e. if the 5′ end is bound to the template surface, the 5′ end of the synthesized oligonucleotide will be bound to the recipient surface, or vice versa. Transfer primers bound at their 5′ ends can bind to the template nucleic acids at their 3′ ends, followed by enzymatic extension to produce nucleic acids complementary to the template oligos and bound to the recipient array surface at their 5′ ends.

In some cases, only full-length template nucleic acid products are used to generate complements on the recipient array. In some cases, at least 30%, 40%, 50%, 60%, 70%, 80%, 90%, 95%, 96%, 97%, 98%, 99%, 99.9% or 100% of template nucleic acid oligos on the template array are full-length products (oligos). In some cases, at least 30%, 40%, 50%, 60%, 70%, 80%, 90%, 95%, 96%, 97%, 98%, 99%, 99.9% or 100% of transfer or recipient nucleic acid products (oligos) generated on the recipient array are full-length products. The generation of partial-length products on the recipient array during ETS can be due to incomplete extension of full-length template oligos during polymerase-driven synthesis. The generation of full-length products on the recipient arrays can be accomplished using AFR as provided herein.

In some cases, the recipient array includes on it primers that hybridize a portion of the template polymers (e.g., oligos) such that extension reactions occur until all of the template polymers (e.g., oligos) are used as templates for synthesis of a complementary recipient oligos on a complementary array (or recipient array). In some instances, synthesis of the recipient array occurs such that on average at least 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, or 50% of the template polymers (e.g., oligos) are used to generate complementary sequences on the recipient array. Stated differently, a recipient array, post-transfer, can comprise recipient nucleotides (e.g., oligos) synthesized using at least 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, or 50% of the template oligonucleotides as templates.

The array transfer process (e.g., ETS) can invert the orientation of the template nucleic acids. That is, if the 5′ end is bound to the template surface, the 3′ end of the synthesized oligonucleotide will be bound to the recipient surface, or vice versa.

Template nucleic acids (e.g., oligos) bound to the template array surface (template surface) at their 3′ ends can hybridize to transfer primers on the recipient array bound to the recipient array surface at their 5′ ends. Enzymatic extension of the transfer primers produces nucleic acids (e.g., oligos) complementary to the template nucleic acids (e.g., oligos) and bound to the recipient array surface at their 5′ ends. In some cases, partial-length oligos in a feature (spot) of the template array) are utilized to generate complementary partial length oligos on a recipient array. In some cases, full-length oligos in a feature (spot) of the template array are utilized to generate complementary full-length oligos on a recipient array.

The template and recipient surfaces can be biocompatible, such as polyacrylamide gels, modified polyacrylamide gels, PDMS, silica, silicon, COC, metals such as gold, chrome, or chromium, or any other biocompatible surface. If the surface comprises a polymer gel layer, the thickness can affect its deformability or flexibility. The deformability or flexibility of a gel layer can make it useful in maintaining contact between surfaces despite surface roughness. Details of the surfaces are further discussed herein.

Reagents and other compounds including enzymes, buffers, and nucleotides can be placed on the surface or embedded in a compatible gel layer. The enzymes can be polymerases, nucleases, phosphatases, kinases, helicases, ligases, recombinases, transcriptases, or reverse transcriptases. In some cases, the enzymes on the surface or embedded in a compatible gel layer comprise a polymerase. Polymerases can include, but are not limited to, PolI, PolII, PolIII, Klenow, T4 DNA Pol, modified T7 DNA Pol, mutated modified T7 DNA Pol, TdT, Bst, Taq, Tth, Pfu, Pow, Vent, Pab, Phusion, pyrophage and others. Details of the surfaces are further discussed herein. In some cases, the enzymes on the surface or embedded in a compatible gel layer comprise a ligase. Ligases can include, but are not limited to, E. coli ligase, T4 ligase, mammalian ligases (e.g., DNA ligase I, DNA ligase II, DNA ligase III, DNA ligase IV), thermostable ligases, and fast ligases.

The surface of the recipient array can be a gel formed on top of the template array. The reaction mixture can be placed on the surface of the recipient array or embedded in a recipient surface. In some cases, the reaction mixture is placed on the surface of the recipient array. In some cases, the reaction mixture is embedded in the recipient surface. The recipient surface can be a compatible gel layer. The reaction mixture can comprise any reagent necessary to conduct enzymatic transfer by synthesis (ETS).

Enzymatic transfer of a template array by ETS can be conducted as follows: 1.) enzyme mix is prepared (e.g., 37 μL H₂O, 5 μL 10× Thermopol buffer, 5 μL of 10 mg/mL BSA, 1 μL of 10 mM dNTPs, and 2 μL of 8 U/μL Bst enzyme); 2.) enzyme mix is applied to a recipient array (e.g., an acrylamide gel coated glass slide with coupled oligonucleotide primers prepared as described elsewhere in this disclosure); 3.) a template array is placed face-to-face with the recipient array and allowed to react (e.g., clamped together in a humidity chamber for 2 hours at 55° C.); 4.) the template and recipient arrays are separated (e.g., loosened by application of 4×SSC buffer and pulled apart with the aid of a razor blade); 5.) the template array is rinsed (e.g., in DI water) and dried (e.g., with N₂); and 6.) the recipient array is rinsed (e.g., with 4×SSC buffer and 2×SSC buffer). In some cases, the oligos on the template array comprise adaptors, such that a bottom adaptor is located proximal to the template array surface, while a top adaptor is located distal from the template array surface. While the sandwich is heated to 55° C., Bst polymerase in Thermopol PCR buffer can extend the primers from the recipient array hybridized to the bottom adaptor of the template array, which can create a dsDNA molecular bridge between the template and recipient array surfaces. Upon physical separation, the second surface (i.e., recipient array) can contain the complementary ssDNA barcode array with the 5′ end of the oligos attached to the surface and the 3′ end available for polymerase extension. Since both the uniformly dispersed primer on the template array and the barcode oligos on the recipient array can be tethered to their respective surfaces, the relative locations of the transferred features can be maintained (in mirror image). To achieve intimate contact and thus uniform transfer over the full chip area, a broad range of surface materials (PDMS, Polyacrylamide), thicknesses, and process conditions can be used. The efficiency of face-to-face transfers can result in reduced density of oligos within each copied array feature. One of skill in the art can appreciate that the transfer conditions can be optimized by, for example, varying the gel transfer conditions, e.g. choice of enzyme, process temperature and time, length of primers, or surface material properties. Alternatively, post-transfer surface amplification via solid-phase PCR (e.g. bridge-PCR) can be used increase the barcode density to the desired level as described herein.

### Oligonucleotide Immobilization Transfer (OIT)

In some instances, the generation of a recipient array is performed by non-enzymatic transfer. One form of non-enzymatic transfer is oligonucleotide immobilization transfer (OIT). In OIT, the template nucleic acids (e.g., oligo) on a template array can be single-stranded. Primers comprising sequence complementary to a portion of the template oligos can hybridize to the template oligos and be extended by primer extension in order to generate and can be made double-stranded template oligos on the template array. The primers used for primer extension can be in solution. Many polymerases can be used for OIT, including PolI, PolII, PolIII, Klenow, T4 DNA Pol, modified T7 DNA Pol, mutated modified T7 DNA Pol, TdT, Bst, Taq, Tth, Pfu, Pow, Vent, Pab, Phusion and others. In some cases, the primers used for primer extension comprise linkers that are used to immobilize or bind strand of the double-stranded template oligo generated by primer extension on a surface of a recipient array. The recipient array surface can be a planar surface, a bead, or a gel as provided herein. In some cases, the recipient array surface is a polyacrylamide gel formed during OIT. In some cases, subsequent to extension, the linkers can be bound to a recipient array surface. The recipient array surface can be any array surface as provided herein such as a polymer gel or modified glass surface. In OIT, the template and recipient array surfaces can be then be separated. The DNA (i.e., double-stranded template oligos) can be melted prior to separation.

In some cases, the primers used in OIT are 5′-acrydite modified primers. The 5′-acrydite modified primers can be capable of incorporation into a polymer gel (e.g., polyacrylamide) during polymerization as provided herein. Extension products from the template nucleic acids (e.g., oligos) can then be generated with the acrydite primers, contacted with a substrate with a binding treatment (e.g., unpolymerized polyacrylamide coating precursor), incorporated during polymerization, and separated. The primers can be 5′-hexynyl-polyT-DNA. In some cases, primer extension products from the template nucleic acids are generated via binding and extension of complementary 5′-hexynyl-polyT-DNA primers. Following extension, the 5′hexynyl-polyT-DNA primers can be: 1) contacted with a substrate with a binding treatment (such as glass treated with silane), 2) linked to a cross-linker such as, for example, a homobifunctional linker such as 1,4-Phenylene Diisothiocyanate (PDITC), 3) linked to an N3 bonding group with a PEG linker, 4) bonded to the substrate at the N3 group, and 5) separated during a second stage of OIT. The surfaces can be any of the surfaces as discussed herein. Other cross-linkers that can be used in place of PDITC can include dimethyl suberimidate (DMS), disuccimidyl carbonate (DSC) and/or disuccimidyl oxylate (DSO). This process can preserve the orientation of the oligonucleotides, i.e. if the 5′ end is bound to the template array surface, the 5′ end of the synthesized oligonucleotide will be bound to the recipient array surface, or vice versa. While enzymatic extension can be used prior to the transfer, the transfer itself can be conducted without enzymatic reactions.

In some cases, an oligo array with 5′ to 3′ orientation can be generated without enzymatic transfer. For example, the unbound end of the synthesized nucleic acid sequences on a template oligo array can comprise a linker sequence complementary to a sequence at or near the array-bound end of the oligo, allowing the oligo to circularize. The oligo can further comprise a restriction sequence at the same end. Digestion of the restriction sequence on circularized oligos serve to flip the full-length oligos containing the linker sequence and cut loose any partial-length oligo products on the array which lack the linker sequence. Many restriction enzymes and their associated restriction sites can be used, including but not limited to EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, SacI, SalI, ScaI, SpeI, SphI, StuI, and XbaI.

Automated Library Preparation

Techniques of the present disclosure can automate sequencing library preparation steps. The libraries can be prepared on a spatially barcoded chip so their relative location in the genome can be determined. The NGS library can then be sequenced using any NGS platform (e.g., Illumina HiSeq).

Once extension products are produced from the target polynucleotide, as described elsewhere in this disclosure, the extension products can be either sequenced directly or used to generate sequencing libraries for subsequent sequencing. In some cases, following processing of a target polynucleotide, a nucleic acid library is produced. The nucleic acid library can be a sequencing library that can be produced from extension products.

In some cases, prior to sequencing, extension products produced by the methods described herein are released from an oligo array. In some cases, the bond between the extension product and the primer substrate can be broken with thermal energy. In some cases, the extension product can be detached from the primer substrate by mechanical breakage or shear. In some cases, the array-bound primers (oligos) may have a restriction site in their 5′ or 3′ end, which is incorporated into the extension product and allows for the selective cleavage and release of the extension products or part thereof. In some cases, releasing an extension product from an oligo array can be via digestion of the extension product with an enzyme for fragmenting nucleic acids as provided herein. In some cases, an extension product is released from an oligo array by digestion with restriction enzymes. The restriction enzymes can be any restriction enzymes known in the art and/or provided herein. In some cases, the extension product is enzymatically cleaved using NEB fragmentase. The digestion time for enzymatic digestion of the extension products can be adjusted to obtain select fragment sizes. In some cases, the extension products can be fragmented into a population of fragmented extension products of one or more specific size range(s).

In some cases, polynucleotide fragments generated by fragmentation of extension products on an oligo array as generated by the methods provided herein are subjected to end repair. End repair can include the generation of blunt ends, non-blunt ends (i.e. sticky or cohesive ends), or single base overhangs such as the addition of a single dA nucleotide to the 3′-end of the double-stranded nucleic acid product by a polymerase lacking 3′-exonuclease activity. In some cases, end repair is performed on the fragments to produce blunt ends wherein the ends of the fragments contain 5′ phosphates and 3′ hydroxyls. End repair can be performed using any number of enzymes and/or methods known in the art. An overhang can comprise about, more than, less than, or at least 1, 2, 3, 4, 5, 6, 7, 8, 9, 10, 11, 12, 13, 14, 15, 16, 17, 18, 19, or 20 nucleotides.

In some cases, extension products generated by the methods provided herein and bound to an oligo array as provided herein, remain bound to the oligo array and a sequencing library is generated from the bound extension products. The generation of a sequencing library from oligo array bound extension products generated by the methods provided herein can be by generating a second set of extension products using the array-bound extension products as templates. These second extension products can comprise a sequence complementary to the barcode sequence. The sequence complementary to the barcode sequence can be correlated to the original barcode sequence and thereby convey the same positional information as the original barcode. The second extension products can also comprise a sequence corresponding to a region or segment of the target polynucleotide, as they can be complementary to the regions of the first extension products that can be complementary to the target polynucleotide from which the array bound extension products were generated

In some cases, preparation of a sequencing library from oligo array bound extension products generated by the methods provided herein is performed by hybridizing non-substrate bound primers (i.e., primers in solution or “free” primers) to the array-bound extension products and extending the hybridized non-substrate bound primers using the array bound extension products as template to generate non-array bound (or free) extension products. The non-substrate bound primers can hybridize to the array-bound extension products, for example through a random sequence segment as described herein of the non-substrate bound primer (e.g., random hexamer, etc.). The random sequence can be at least 5, 6, 7, 8, 9, 10, 11, 12, 13, 14, or 15 base pairs or nucleotides. The random sequence can be at most 5, 6, 7, 8, 9, 10, 11, 12, 13, 14, or 15 base pairs or nucleotides. Free primers can comprise PCR primer sequences. PCR primer sequences can be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 base pairs or nucleotides. PCR primer sequences can be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 base pairs or nucleotides. The non-substrate bound primers can comprise adaptor sequences. The adaptor sequences can be compatible with any sequencing platforms known in the art. In some cases, the adaptor sequence comprises sequence compatible for use in Illumina NGS sequencing methods such as the Illumina HiSeq 2500 system. The adaptor sequences can be Y-shaped adaptor, or duplex or partial duplex adaptors. Extension of the non-substrate bound primers hybridized to the array bound extension products can be conducted with enzymes, such as DNA polymerase. The polymerase can include, but are not limited to, PolI, PolII, PolIII, Klenow, T4 DNA Pol, modified T7 DNA Pol, mutated modified T7 DNA Pol, TdT, Bst, Taq, Tth, Pfu, Pow, Vent, Pab, and Phi-29. For example, extension reactions can be conducted using Bst polymerase by incubating the template nucleic acid and primers with Bst polymerase and dNTPs at 65° C. in 1× Isothermal Amplification Buffer (e.g., 20 mM Tris-HCl, 10 mM (NH₄)₂SO₄, 50 mM KCl, 2 mM MgSO₄, and 0.1% Tween 20).

Non-array bound extension products generated by the methods provided herein can comprise sequence corresponding to a segment of the target polynucleotide. That is, a non-array bound extension product can comprise sequence complementary to some or all of the segment of an array-bound extension product from which it was generated which can comprise sequence corresponding to or complementary to a segment of the target polynucleotide. A non-array bound extension product can comprise a barcode which comprises sequence complementary to the barcode sequence of the array-bound extension product. This complementary barcode can convey the same positional information conveyed by the original barcode sequence by correlating the complementary barcode sequence with the original barcode sequence. In a non-array bound extension product, the positional information conveyed by the barcode or complementary barcode can be correlated with the sequence corresponding to a segment of the target polynucleotide, thereby locating the segment of the target polynucleotide along the length of the stretched target polynucleotide molecule. Non-array bound extension products can comprise one or more PCR primer sequences. A non-array bound extension product can comprise a PCR primer sequence complementary to a PCR primer sequence in the array-bound extension product from which it was generated. A non-array bound extension product can comprise a PCR primer sequence from the non-array bound primer that was extended to generate the non-array bound extension product. Non-array bound extension products can comprise adaptor sequences, such as sequencing adaptors. In some cases, an adaptor sequence appended to a non-array bound extension product comprise sequence compatible for use in Illumina NGS sequencing methods such as the Illumina HiSeq 2500 system.

Extension products (non-array bound or released from an oligo array as described herein) or fragments thereof can be amplified and/or further analyzed such as by sequencing. The sequencing can be any sequencing methods known in the art. Amplification can be conducted by any amplification methods known in the art or provided herein. Amplification can be conducted with any enzyme as provided herein. For example, reactions can be conducted using Bst polymerase by incubating the template nucleic acid and primers with Bst polymerase and dNTPs at 65° C. in 1× Isothermal Amplification Buffer (e.g., 20 mM Tris-HCl, 10 mM (NH₄)₂SO₄, 50 mM KCl, 2 mM MgSO₄, and 0.1% Tween 20). Amplification can utilize PCR primer sites incorporated into the extension products, for example from the array-bound primers (oligos) and the non-substrate bound primers. Amplification can be used to incorporate adaptors, such as sequencing adaptors, into the amplified extension products. The sequencing adaptors can be compatible with any sequencing method known in the art.

Library Amplification.

The polynucleotide molecule can be sequenced on a sequencer (such as the Illumina HiSeq). The molecules can be obtained by performing linear amplification with primers directed toward a distal primer site on the immobilized molecule. However, if needed, an amplification reaction (e.g., PCR) can be performed on the chip bound DNA molecules for exponential amplification of the library.

Bioinformatics and Software

After sequencing, the sequence data can be aligned. Each sequence read can be separated into primer/tag sequence information, based on the known designed sequences of the primers/tags, and target polynucleotide information. Alignment can be aided by the encoded positional barcode information associated with each piece of target polynucleotide through its primer/tag sequence. Sequencing of the sequencing library or released extension products can generate overlapping reads with the same or adjacent barcode sequences. For example, some extension products can be long enough to reach the next specific sequence site associated with the target polynucleotide. Use of barcode sequence information can group together likely overlapping reads, which can increase accuracy and reduce computational time or effort.

In some cases, sequence reads and associated barcode sequence information obtained by the methods provided herein are analyzed by software. The sequence reads can be short (e.g., <100 bps) or long sequence reads (e.g., >100 bps). The software can perform the steps of arranging sequence reads derived from the same template. These reads can be identified by, for example, searching for reads that have barcodes from the same or neighboring columns in an oligo array comprising spot or regions as provided herein. In some cases, only reads of a certain range of distance, horizontal rows, and/or vertical columns are considered as putatively from the same template. In reading the barcodes, the software can take into account potential sequencing (and other) errors based upon barcode design. The error can be barcodes with edit distance four allows certain errors. In some cases, if a barcode contains too many errors and cannot be uniquely identified, its associate read is not directly used to assemble a sequence. While many reads can be assembled based upon relative barcode position (e.g., row numbers), some gaps can be filled by aligning reads coming from the same genomic region.

For assembly of sequence reads based on comparison to a reference DNA sample (e.g. genome), such as in re-sequencing, software useful for re-sequencing assembly can be used. The software used can be compatible with the type of sequencing platform used. If sequencing is done with an Illumina system, software packages such as Partek, Bowtie, Stampy, SHRiMP2, SNP-o-matic, BWA, BWA-MEM, CLC workstation, Mosaik, Novoalign, Tophat, Splicemap, MapSplice, Abmapper. ERNE-map (rNA), and mrsFAST-Ultra can be used. For SOliD based NGS sequencing, Bfast, Partek, Mosaik, BWA, Bowtie, and CLC workstation can be used. For 454 based sequencing, Partek, Mosaic, BWA, CLC workstation, GSMapper, SSAHA2, BLAT, BWA-SW, and BWA-MEM can be used. For Ion torrent based sequencing, Partek, Mosaic, CLC workstation, TMAP, BWA-SW, and BWA-MEM can be used. For de novo assembly of sequence reads obtained from the methods provided herein, any alignment software known in the art can be used. The software used can use an overlap layout approach for longer reads (i.e., >100 bps) or a de Bruijn graph based k-mer based approach for shorter reads (i.e., <100 bp reads). The software used for de novo assembly can be publically available software (e.g., ABySS, Trans-ABySS, Trinity, Ray, Contrail) or commercial software (e.g., CLCbio Genomics Workbench).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising:
a) contacting a biological sample comprising a plurality of biological molecules with a spatial barcode array, wherein the spatial barcode array comprises a plurality of oligonucleotides attached thereto, wherein each of the plurality of oligonucleotides comprises a barcode sequence that identifies a location of the plurality of oligonucleotides on the spatial barcode array;
b) attaching the plurality of oligonucleotides to the plurality of biological molecules to generate a plurality of tagged biological molecules;
c) sequencing at least a portion of the plurality of tagged biological molecules; and
d) determining a location of the plurality of biological molecules within the biological sample based on the barcode sequence attached to the tagged biological molecules.

2. The method of claim 1, wherein the plurality of biological molecules are DNA.

3. The method of claim 1, wherein the plurality of biological molecules are RNA.

4. The method of claim 3, wherein the RNA is mRNA.

5. The method of claim 4, further comprising, prior to c), reverse transcribing the mRNA to cDNA.

6. The method of claim 5, wherein the plurality of oligonucleotides comprise a polyT sequence.

7. The method of claim 1, wherein the attaching comprises ligating the plurality of oligonucleotides to the plurality of biological molecules.

8. The method of claim 1, wherein the attaching comprises annealing the plurality of oligonucleotides to the plurality of biological molecules.

9. The method of claim 8, further comprising, after the annealing, extending the plurality of oligonucleotides, using the plurality of biological molecules as a template, to generate a sequencing library.

10. The method of claim 1, further comprising, prior to the sequencing, amplifying the plurality of tagged biological molecules to generate an amplified sequencing library

11. The method of claim 1, wherein each of the plurality of oligonucleotides comprises one or more adaptor sequences.

12. The method of claim 1, wherein each of the plurality of oligonucleotides comprises one or more primer sequences.

13. The method of claim 1, wherein the barcode sequence identifies an x and y coordinate for the plurality of biological molecules within the biological sample.

14. The method of claim 1, wherein the biological sample is a tissue section or a transfer of a tissue section.

15. The method of claim 14, further comprising, performing a)-d) on a plurality of consecutive tissue sections to generate a three-dimensional profile of the biological molecules within the biological sample.

16. The method of claim 15, wherein the barcode sequence further identifies a z coordinate for the plurality of biological molecules within the three-dimensional profile.

17. The method of claim 14, wherein the tissue section is a biopsy sample.

18. The method of claim 14, wherein the tissue section is a formalin-fixed paraffin-embedded (FFPE) tissue section.

19. The method of claim 1, wherein the barcode sequence of each of the plurality of oligonucleotides is different.

20. The method of claim 1, wherein the barcode sequence is indicative of the location of an oligonucleotide of the plurality of oligonucleotides on the spatial barcode array to within 2 μm.

21. The method of claim 1, wherein the barcode sequence is indicative of the location of an oligonucleotide of the plurality of oligonucleotides on the spatial barcode array to within 1 μm.

22. The method of claim 1, wherein the barcode sequence is indicative of the location of an oligonucleotide of the plurality of oligonucleotides on the spatial barcode array to within 0.5 μm.

23. The method of claim 1, wherein the barcode sequence is indicative of the location of an oligonucleotide of the plurality of oligonucleotides on the spatial barcode array to within 0.2 μm.

24. The method of claim 1, wherein the barcode sequence is indicative of the location of an oligonucleotide of the plurality of oligonucleotides on the spatial barcode array to within 0.1 μm.

25. The method of claim 1, wherein the spatial barcode array comprises a solid support.

26. A method comprising:
a) contacting a biological sample comprising a plurality of biological molecules with a spatial barcode array, wherein the spatial barcode array comprises a plurality of oligonucleotides attached thereto, wherein each of the plurality of oligonucleotides comprises a barcode sequence that identifies a location of the plurality of oligonucleotides on the spatial barcode array;
b) attaching the plurality of oligonucleotides to a signal sequence associated with each of the plurality of biological molecules to generate a plurality of tagged signal sequences;
c) sequencing at least a portion of the plurality of tagged signal sequences; and
d) determining a location of the plurality of biological molecules within the biological sample based on the barcode sequence attached to the plurality of tagged signal sequences.

27. The method of claim 26, wherein the plurality of biological molecules are proteins.

28. The method of claim 26, wherein the signal sequence is a tag oligonucleotide.

29. The method of claim 26, wherein the signal sequence is conjugated to an affinity molecule.

30. The method of claim 29, wherein the affinity molecule is an antibody, an aptamer, a peptide or a peptidomimetic.

31. The method of claim 29, further comprising, prior to b), contacting the biological sample with a plurality of affinity molecules, each of which are conjugated to a signal sequence, under conditions that permit binding of the plurality of affinity molecules to the plurality of biological molecules.

32. The method of claim 29, wherein at least a portion of the signal sequence identifies the affinity molecule conjugated thereto.

33. The method of claim 29, wherein each affinity molecule is conjugated to a different signal sequence.

34. The method of claim 26, wherein the attaching comprises ligating the plurality of oligonucleotides to the signal sequence associated with each of the plurality of biological molecules.

35. The method of claim 26, wherein the attaching comprises annealing the plurality of oligonucleotides to the plurality of signal sequences associated with each of the plurality of biological molecules.

36. The method of claim 35, further comprising, after the annealing, extending the plurality of oligonucleotides, using a signal sequence associated with each of the plurality of biological molecules as a template to generate a sequencing library.

37. The method of claim 26, further comprising, prior to the sequencing, amplifying the plurality of tagged signal sequences to generate an amplified sequencing library.

38. The method of claim 26, wherein each of the plurality of oligonucleotides comprises one or more adaptor sequences.

39. The method of claim 26, wherein each of the plurality of oligonucleotides comprises one or more primer sequences.

40. The method of claim 26, wherein the barcode sequence identifies an x and y coordinate for the plurality of biological molecules within the biological sample.

41. The method of claim 26, wherein the biological sample is a tissue section or a transfer of a tissue section.

42. The method of claim 41, further comprising, performing a)-d) on a plurality of consecutive tissue sections to generate a three-dimensional profile of the plurality of biological molecules within the biological sample.

43. The method of claim 42, wherein the barcode sequence further identifies a z coordinate for the plurality of biological molecules within the three-dimensional profile.

44. The method of claim 41, wherein the tissue section is a biopsy sample.

45. The method of claim 41, wherein the tissue section is a formalin-fixed paraffin-embedded (FFPE) tissue section.

46. The method of claim 26, wherein the barcode sequence of each of the plurality of oligonucleotides is different.

47. The method of claim 26, wherein the barcode sequence is indicative of the location of an oligonucleotide of the plurality of oligonucleotides on the spatial barcode array to within 2 μm.

48. The method of claim 26, wherein the barcode sequence is indicative of the location of an oligonucleotide of the plurality of oligonucleotides on the spatial barcode array to within 1 μm.

49. The method of claim 26, wherein the barcode sequence is indicative of the location of an oligonucleotide of the plurality of oligonucleotides on the spatial barcode array to within 0.5 μm.

50. The method of claim 26, wherein the barcode sequence is indicative of the location of an oligonucleotide of the plurality of oligonucleotides on the spatial barcode array to within 0.2 μm.

51. The method of claim 26, wherein the barcode sequence is indicative of the location of an oligonucleotide of the plurality of oligonucleotides on the spatial barcode array to within 0.1 μm.

52. The method of claim 26, wherein the spatial barcode array comprises a solid support.
